(19) United States
(12) Patent Application Publication
(10) Pub. No.: US 2012/0070449 A1
(43) Pub. Date: Mar. 22, 2012

(54) METHODS OF TREATING INFLAMMATION

(75) Inventors: Idit Shachar, Ramat-Gan (IL); Einat Toister, Rehovot (IL)

(73) Assignee: Yeda Research And Development Co. Ltd., Rehovot (IL)

(21) Appl. No.: 13/322,455

(22) PCT Filed: May 27, 2010
(86) PCT No.: PCT/IL10/00420

§ 371 Date: Nov. 24, 2011

Related U.S. Application Data
(60) Provisional application No. 61/213,315, filed on May 28, 2009.

(51) Int. Cl. A61K 39/395; A61P 31/00; A61P 3/10; A61P 29/00; A61K 38/16; A61K 38/08; A61P 37/08; A61K 38/00; A61P 35/00

(52) U.S. Cl. 424/172.1; 514/1.1; 514/2.3; 514/6.9; 514/19.3; 514/21.2; 514/21.6

(57) ABSTRACT

A method of treating an inflammation in a subject in need thereof is disclosed. The method comprising administering to the subject an agent capable of down-regulating an activity or expression of CD151 in a lymphocyte, with the proviso that the agent is not CCL2, thereby treating the inflammation.

### FIELD AND BACKGROUND OF THE INVENTION

The present invention, in some embodiments thereof, relates to methods of treating inflammation and more particularly, but not exclusively to down regulation of CD151 for the treatment of inflammation.

The surveillance of the body for foreign antigens is a critical function of the immune system. B and T lymphocytes develop in the primary lymphoid organs while their capabilities are employed throughout the body. Accordingly, B and T lymphocytes continually patrol the body for foreign antigens by circulating from the blood, through tissues, into lymph nodes (LNs), and back to the blood. Lymphocyte migration out of the vasculature is therefore a key regulatory point for controlling immune responses. It is controlled by multi-step processes that include chemoattraction, cell-cell adhesion and transmigration through cell layers. The continuous recirculation of lymphocytes and their movement from the blood into peripheral tissues is a central feature of immune surveillance, but also contributes to the pathogenesis of inflammatory diseases, autoimmune diseases, as well as the development and progression of allergic responses.

VLA4, a cell surface heterodimer in the integrin superfamily of adhesion receptors, is expressed on several different circulating blood cell types, including T cells. VLA4 is involved in cell-cell interactions and mediates cell attachment to vascular cell adhesion molecule-1 (VCAM-1), which is expressed on the surface of endothelial cells as a result of their exposure to cytokines. The VLA-4-VCAM-1 interaction is responsible, at least in part, for recruitment of T cells to areas of vascular inflammation and plays an essential role during various types of inflammation [Lobb and Hemler, J Clin Invest (1994) 94(5): 1722-8; Postigo et al., Res Immunol (1993) 144(9): 723-35; discussion 754-62]. VLA-4 also binds to fibronectin on the extracellular matrix (ECM) and this interaction is important for migration of T lymphocytes to antigenic sites [Mannion et al., J Immunol (1996) 157(5): 2039-47; Yuan et al., Biochem J (1996) 318 (Pt 2): 591-6], thus VLA-4 expression is critical for the migration of T lymphocytes into inflamed tissue.

The protein CD151 (PETA-3/SFA-1), a member of the tetraspanins family (also known as the transmembrane 4 superfamily), is expressed in various cell types including epidermal basal cells, epithelial cells, skeletal, smooth and cardiac muscle, endothelial cells, T cells, neutrophils, platelets, and schwann cells and has a strong molecular association with the β1 family of integrins. CD151 possesses four highly conserved transmembrane domains, cytoplasmic amino and carboxyl termini, and two extracellular loops (FIG. 5), the larger of which contains the distinctive pattern of cysteine residues that help to define the family. It was previously shown in human melanoma cells that homophilic interactions between CD151 proteins on the surface of neighboring cells increase c-Jun activity through the activation of FAK, Src, p38 MAPK, and JNK, leading to enhanced cell motility and MMP-9 expression [Hong et al., J Biol Chem (2006) 281(34): 24279-92]. Although the physiological function of CD151 is largely unknown, in vitro functional studies showed that CD151 is involved in cell adhesion, motility, and polarity [Yauch et al., Mol Biol Cell (1998) 9(10): 2751-65]. It was shown that antibodies to either CD151 or α3β1 lead to a 88-92% reduction in neutrophil motility in response to f-Met-Leu-Phe on fibronectin, suggesting a functionally important role of these complexes in cell migration [Yauch et al., Mol Biol Cell (1998) 9(10): 2751-65]. Since cellular processes regulated by CD151 (such as cell adhesion, migration, and spreading) are integrin-mediated adhesive events, it has been proposed that CD151 modulates integrin activity and function, such as those of β1 integrins [Nishiuchi et al., Proc Natl Acad Sci USA (2005) 102(6): 1939-44].

Hasegawa et al. have reported that the expression level of CD151 on adult T cell leukemia (ATL) cells from the lymph nodes of lymphoma-type ATL patients were significantly higher than those on circulating ATL cells [Hasegawa et al., J of immunology (1998) 161: 3087-3095]. Thus, they suggest that increased expression of CD151 may act to retain ATL cells in the lymph nodes.

As was previously reported [Flaishon et al., Blood (2008) 112: 5016-5025; PCT Publication No. WO 2008/012796], picomolar [pM] levels of circulating CCL2 (also called Macrophage Chemotactic Protein-1 or MCP-1) can exert global suppressive effects on T-cell trafficking and differentiation within peripheral lymph nodes. The impaired homing of T lymphocytes to the peripheral lymph nodes by CCL2 results in attenuated progression of both asthma and adjuvant arthritis. Thus, low levels of circulating CCL2 can exert global suppressive effects on T-cell trafficking and differentiation within peripheral lymph nodes, and may be clinically beneficial as an anti-inflammatory agent.

PCT Publication No. WO 2007/146968 discloses multivalent binding peptides, including bi-specific binding peptides, having an immunoglobulin effector function. The cell target of the peptide may be an angiogenesis target (e.g. CD151) and the cell may be a T cell. Methods for using such peptides to treat, prevent or ameliorate symptoms of a variety of diseases, disorders or conditions (e.g. inflammatory and autoimmune conditions) are also provided. For example, WO 2007/146968 contemplates targeted recruitment of effector cells (e.g. cytotoxic T lymphocytes) or to localizing therapeutic compounds (e.g. radiolabeled proteins) to cells, tissues, agents and foreign objects to be destroyed or sequestered (e.g. cancer cells).

### SUMMARY OF THE INVENTION

According to an aspect of some embodiments of the present invention there is provided a method of treating an inflammation in a subject in need thereof, the method comprising administering to the subject an agent capable of downregulating an activity or expression of CD151 in a lymphocyte, with the proviso that the agent is not CCL2, thereby treating the inflammation.

According to an aspect of some embodiments of the present invention there is provided a use of an agent capable of downregulating an activity or expression of CD151 in a lymphocyte, with the proviso that the agent is not CCL2, for the manufacture of a medicament identified for treating an inflammation.

According to an aspect of some embodiments of the present invention there is provided a use of an agent capable of downregulating an activity or expression of CD151 in a lymphocyte, with the proviso that the agent is not CCL2, for treating an inflammation.

According to an aspect of some embodiments of the present invention there is provided a pharmaceutical composition comprising as an active ingredient an agent capable of downregulating an activity or expression of CD151 in a lymphocyte, with the proviso that the agent is not CCL2, and a pharmaceutically acceptable carrier.

According to an aspect of some embodiments of the present invention there is provided an isolated peptide comprising an amino acid sequence as set forth in SEQ ID NO: 129.

According to some embodiments of the invention, the agent is an antibody. According to some embodiments of the invention, the antibody is a CD151 blocking antibody.

According to some embodiments of the invention, the CD151 blocking antibody targets the peptide having an amino acid sequence set forth in SEQ ID NO: 129.

According to some embodiments of the invention, the antibody is a VLA4 blocking antibody.

According to some embodiments of the invention, the antibody is a LFA1 blocking antibody.

According to some embodiments of the invention, the agent is a nucleic acid agent suitable for silencing expression in a targeted manner.

According to some embodiments of the invention, the nucleic acid agent is selected from the group consisting of an siRNA, an antisense, a ribozyme and a DNAzyme.

According to some embodiments of the invention, the agent is a peptide agent.

According to some embodiments of the invention, the peptide agent is a CD151 derived peptide capable of blocking dimerization of the CD151 or an interaction thereof with a downstream effector.

According to some embodiments of the invention, the peptide agent has an amino acid sequence set forth in SEQ ID NO: 129.

According to some embodiments of the invention, the subject is a human subject.

According to some embodiments of the invention, the inflammation is associated with a medical condition selected from the group consisting of a cancer, an autoimmune disease, a hypersensitivity, a diabetes, an infectious disease, a transplantation associated disease and an allergy.

According to some embodiments of the invention, the lymphocyte is a T cell.

According to some embodiments of the invention, downregulating an activity or expression of CD151 inhibits lymphocyte migration or homing.

According to some embodiments of the invention, the migration or homing is to a lymphoid organ.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H depict downregulation of CD151 expression by pM levels of CCL2. FIGS. 1A-B, T cells derived from control mice (CCR2 expressing mice) were pre-incubated in the presence or absence of CCL2 (1 ng/ml) for 1 hour and stimulated with CCL21 (100 ng/ml). After fixation, actin polymerization was analyzed as described in the Examples section. FIG. 1A shows the arbitrary values of the actin polymerization. FIG. 1B shows calculation of fold change in actin polymerization. The results presented were an average of four independent experiments; FIGS. 1C-D, control (FIG. 1C) or CCR2−/− (FIG. 1D) T cells were pre-incubated in the presence or absence of CCL2 (1 ng/ml) for several time periods (as indicated in the figures) and RNA was purified. RT-PCR was performed as described in the Examples section. The intensity of the CD151 band following each treatment was normalized by dividing by the measured intensity of the HPRT band from the same treatment. The results presented were representative of three different experiments; FIGS. 1E-G, control (FIGS. 1E-F) or CCR2−/− (FIG. 1G) T cells were pre-incubated in the presence or absence of CCL2 (1 ng/ml) for several time periods (as indicated in the figures). After incubation, the cells were double stained with anti-CD3 and anti-CD151 antibodies. Histogram shows CD151 expression on T cells in cells treated or untreated with CCL2 for 8 hours (FIG. 1E). % of T cells expressing CD151 following treatment with CCL2 for up to 10 hours (FIGS. 1F-G), the graphs summarized the results of three separate experiments. FIG. 1H, T cells were pre-incubated in the presence or absence of CCL2 (1 ng/ml) for several time periods (as indicated in the figure) and RNA was purified. Quantitative Real time PCR was performed using primers for C151 and Actin (as described in the Examples section). Results are expressed as fold change in CD151 mRNA expression relative to actin levels.

FIGS. 2A-H depict CD151 regulation of T cells' cytoskeleton rearrangement and cell migration in vitro and in vivo. FIG. 2A, T cells were incubated with either an activating anti-CD151 antibody or an isotype control (IgG) antibody for different time periods (as indicated in the figure). RNA was purified and RT-PCR was performed as described in the Examples section. The results shown represent three independent experiments with similar results; FIGS. 2B-D, T cells were pretreated with an activating anti-CD151 antibody or with an isotype control (IgG) antibody for 1 hour and were then stimulated with CCL21. FIG. 2B shows actin polymerization (the results shown represent the average of three independent experiments). FIGS. 2C-D depict migration towards the chemokine CCL21 residing in the lower part of the apparatus through FN-uncoated (FIG. 2C) or FN-coated (FIG. 2D) wells as was measured after 3 hours by FACSort. The graphs show an average of three independent experiments; FIGS. 2E-G, T cells were pretreated with a blocking anti-CD151 antibody or with an isotype control (IgG) antibody for 1 hour and stimulated with CCL21. FIG. 2E shows actin polymerization (the results shown represent the average of three independent experiments with similar results). FIG. 2F-G depict migration towards the chemokine CCL21 residing in the lower part of the apparatus through FN-uncoated (FIG. 2F) FN-coated (FIG. 2G) as was measured after 3 hours by FACSort. The results shown represent the average of three independent experiments with similar results; FIG. 2H, FITC-labeled T cells were incubated with a blocking anti-CD151 antibody or an isotype control (IgG) antibody for 1 hour and injected into mice. After 3 hours, the PLNs were collected and the FITC-positive population was analyzed by FACS. The results presented are representative of three separate experiments.

FIGS. 3A-F depict the CD151 complex formation with integrins in T cells and its downstream signaling cascade. FIGS. 3A-B, control T cells were lysed. Following anti-CD151 or anti-isotype control immunoprecipitation, proteins were separated on 8% (w/v) SDS-PAGE and transferred onto nitrocellulose. β1 (FIG. 3A) or α_{L} (FIG. 3B) integrins were detected by Western blot analysis. The results presented are representative of at least five or three different experiments, respectively; FIGS. 3C-E, T cells were pretreated with either an activating anti-CD151 antibody or an isotype control IgG antibody for several time periods. Immediately after stimulation, cells were washed and fast frozen in liquid N2. FIG. 3C, cells were lysed and an aliquot was reserved for total β-catenin analysis. Phosphorylated proteins from the remaining lysate were immunoprecipitated (IP) with an anti-Tyr(P) antibody. Immunoprecipitates and total lysate proteins were separated on 8% (w/v) SDS-PAGE and blotted with an anti-β-catenin antibody. The results shown represent the results of four separate experiments. FIG. 3D, cells were lysed and an aliquot was reserved for total Vav analysis. Phosphorylated proteins from the remaining lysate were immunoprecipitated (IP) with an anti-Tyr(P) antibody. Immunoprecipitates and total lysate proteins were separated on 10% (w/v) SDS-PAGE and blotted with an anti-Vav antibody. The results shown represent three separate experiments. FIG. 3E, cells were lysed and the lysates were separated by 12% (w/v) SDS-PAGE and blotted with anti p-ERK and ERK antibodies. The results shown represent four separate experiments. FIG. 3F, T cells were incubated with 10 μg/ml blocking anti-CD151 antibody (BD pharmingen) or with a control antibody anti-IgG (ImmunoResearch Labs.inc). Phosphorylated proteins from the remaining lysate were immunoprecipitated (IP) with an anti-Tyr(P) antibody. Immunoprecipitates and total lysate proteins were separated on 10% (w/v) SDS-PAGE and blotted with an anti-Vav antibody. The results shown represent three separate experiments.

FIGS. 4A-F depict CCL2 interference with the signals transmitted by CD151. FIGS. 4A-B, control (FIG. 4A) or CCR2−/− (FIG. 4B) T cells were pre-incubated in the presence or absence of CCL2 for several time periods. After immunoprecipitation with an anti-CD151 antibody or an anti-isotype control antibody, proteins were separated on 8% (w/v) SDS-PAGE and transferred onto nitrocellulose. β1 integrin was detected by Western blot analysis. The results presented are representative of at least three different experiments; FIGS. 4C-D, control (FIG. 4C) or CCR2−/− (FIG. 4D) T cells were pre-incubated in the presence or absence of CCL2 for 10 minutes. Cells were then stimulated with either activating anti-CD151 or isotype control (IgG) antibodies for 1 hour. Cells were then stimulated with CCL21, fixed and their actin polymerization was examined. The results shown represent the average of three independent experiments; FIG. 4E, Control T cells were pre-incubated in the presence or absence of CCL2 for 20 hours. Cells were then stimulated with either activating anti-CD151 or isotype control (IgG) antibodies for 1 hour. The cells were then stimulated with CCL21, fixed and their actin polymerization was followed. The graph shows an average of three independent experiments; FIG. 4F, T cells were pre-incubated for 10 minutes with CCL2 and were then stimulated with anti-CD151 activating antibody or an isotype control IgG antibody. Immediately after stimulation, cells were washed and fast frozen in liquid N2. Cells were lysed and an aliquot was reserved for total β-catenin analysis. Phosphorylated proteins from the remaining lysate were immunoprecipitated (IP) with an anti-Tyr(P) antibody. Immunoprecipitates and total lysate proteins were separated on 8% (w/v) SDS-PAGE and blotted with an anti-β-catenin antibody. The results shown represent the results of three separate experiments.

FIGS. 4G-J depict the effect of CD151 inhibition on LFA-1 mediated adhesion to ICAM-1 triggered by CCL21 and adhesion strengthening in mouse T cells. FIGS. 4G-H illustrate mouse T cells pre-treated with 1 ng/ml mCCL2; FIGS. 4I-J illustrate mouse T cells pre-treated with a CD 151 inhibitor antibody.

FIG. 5 is a schematic illustration of the CD151 protein. CD151 comprises four highly conserved transmembrane domains, cytoplasmic amino and carboxyl termini, and two extracellular loops, the larger of which contains the distinctive pattern of cysteine residues that help to define the family.

FIG. 6 is a schematic illustration of the role of CD151 in facilitating T cell migration and homing in a CCL2 dependent manner. As illustrated, CD151 forms a complex with VLA4, essential for transmitting a signaling cascade that involves phosphorylation of β-catenin and Vav, leading to cytoskeleton rearrangement and migration of T cells. pM levels of CCL2, which bind to the CCR2 receptor, have both short and long term effects on CD151. In the short term effect, CCL2 immediately dissociates the CD151/VLA4 complex, resulting in an immediate inhibition of CD151-induced signaling cascade and downregulation of actin cytoskeleton and migration. After few hours, CCL2 downregulates CD151 mRNA expression and as a result downregulates CD151 cell surface expression.

FIG. 7 depicts full length and extracellular domain constructs purification of CD151.

FIGS. 8A-B are schematic illustrations of full length (FL) and extracellular domain (ECD) constructs of CD151 and purification thereof. FIG. 8A depicts the process of CD151 FL/ECD transformation into bacteria, the colony expansion and the plasmid purification; FIG. 8B depicts the transfection of the plasmids into 293 cells.

### DESCRIPTION OF SPECIFIC EMBODIMENTS OF THE INVENTION

The present invention, in some embodiments thereof, relates to methods of treating inflammation and more particularly, but not exclusively to down regulation of CD151 for the treatment of inflammation.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have uncovered that CD151 expression in T cells plays a key role in T cell migration and homing to the lymph nodes, and as such, downregulation of CD151 can be used as an efficient anti-inflammatory modality.

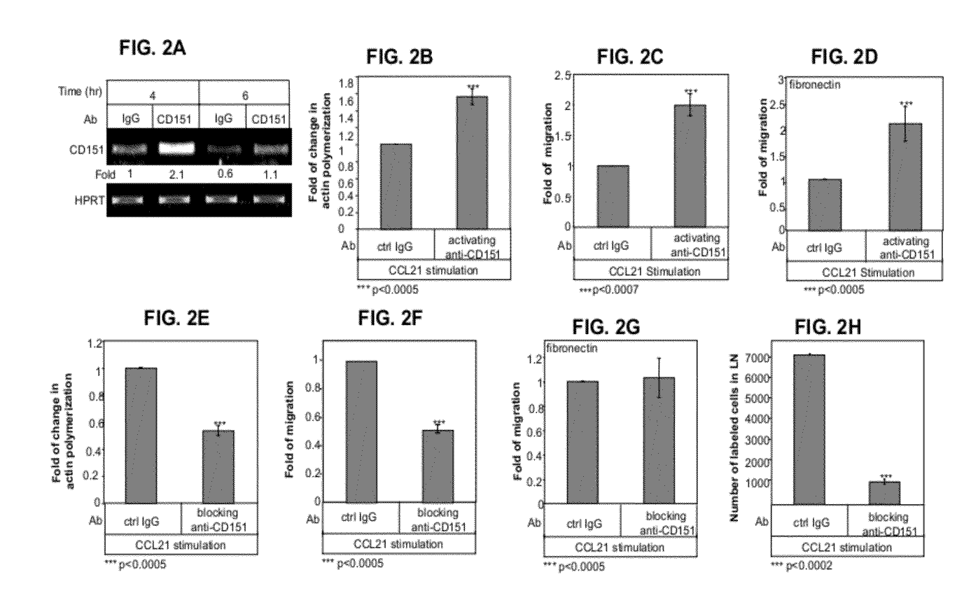

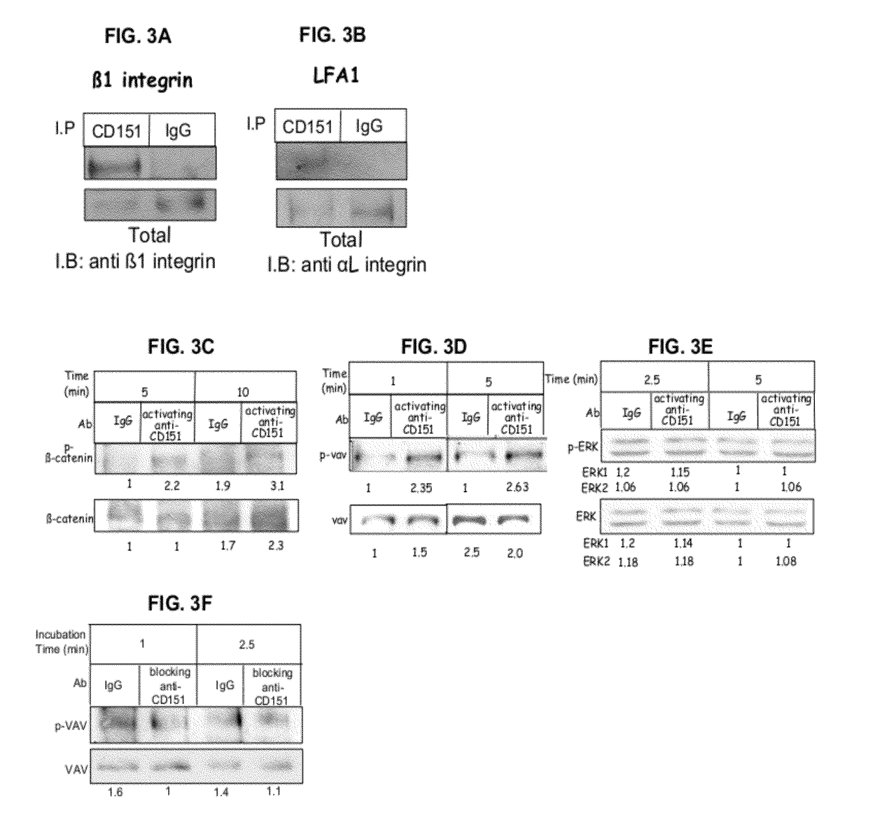

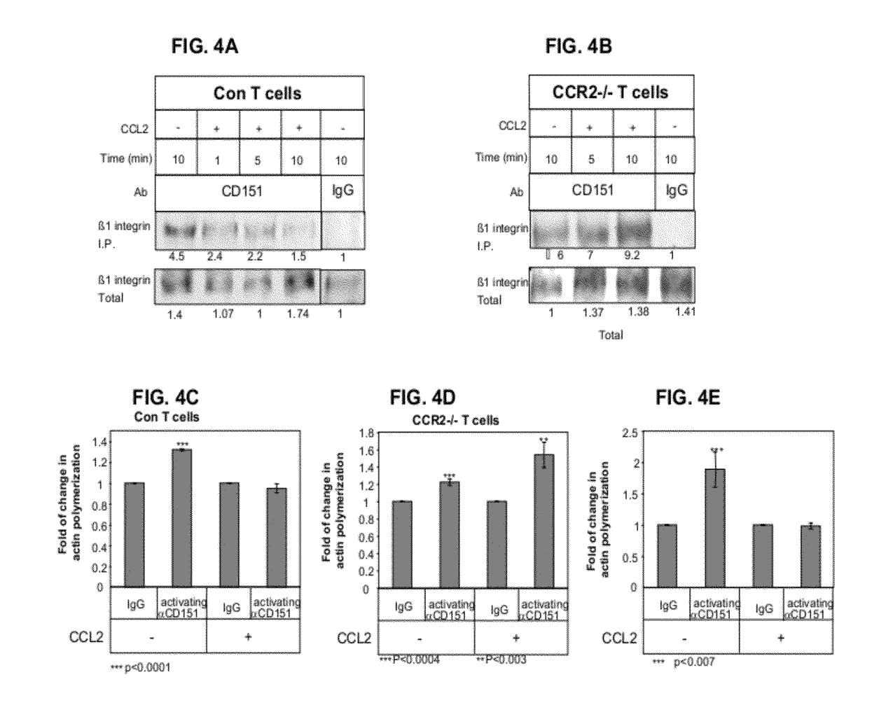

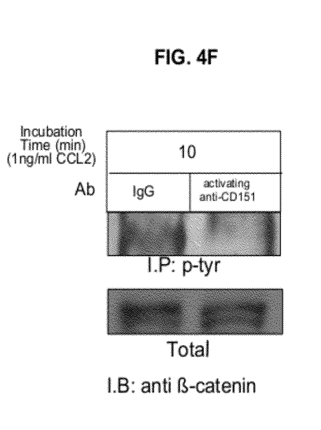

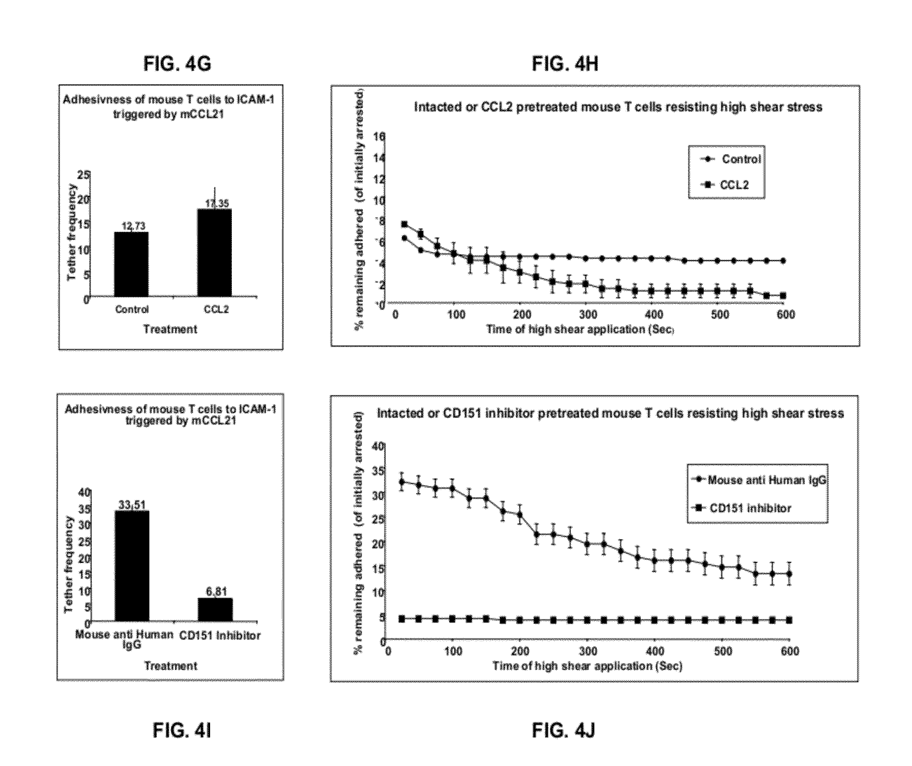

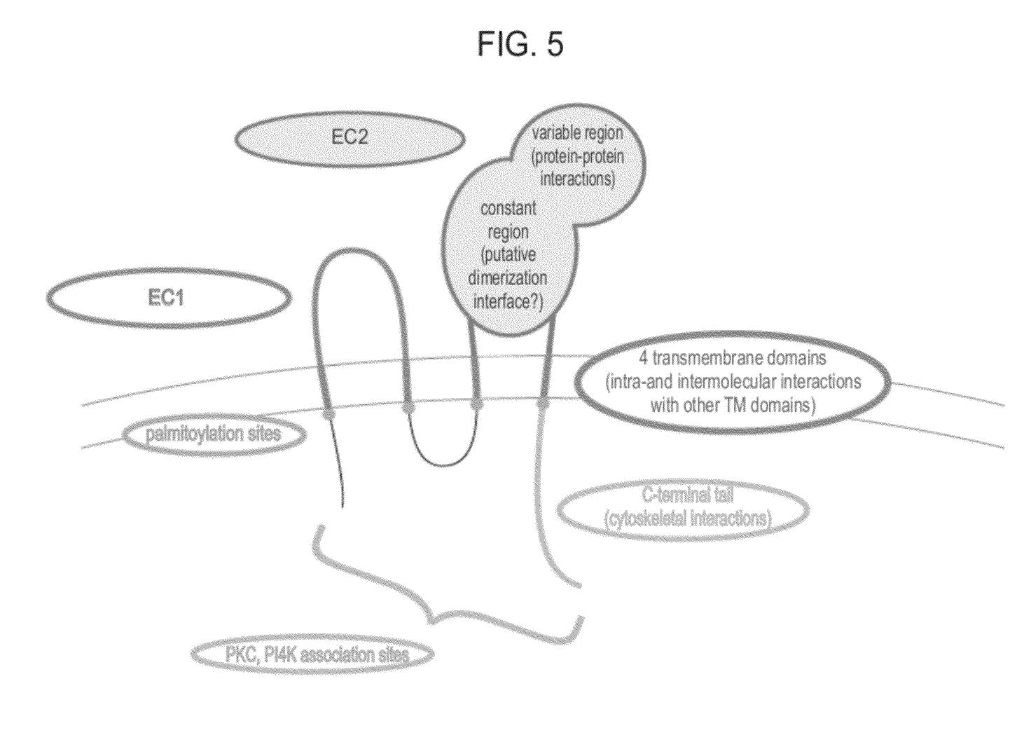

As is shown hereinbelow and in the Examples section which follows, the present inventors have uncovered through laborious experimentation that in T lymphocytes, CD151 regulates T cell cytoskeleton rearrangement and migration (FIG. 2B-D). Following activation of T cells, CD151 forms a complex with the integrin VLA4 (FIG. 3A). The CD151-VLA4 complex initiates a signaling cascade that involves phosphorylation of β-catenin (FIG. 3B) and Vav (FIG. 3C), leading to cytoskeleton rearrangement and migration of the cells. According to the present teachings, picomolar (pM) levels of CCL2 has both short and long term downregulatory effects on CD151. In the short term effect, CCL2 immediately dissociated the CD151/VLA4 complex (FIG. 4B), resulting in an immediate inhibition of CD151-induced signaling cascade and downregulation of actin cytoskeleton and migration (FIG. 4C). After a few hours, CCL2 downregulates CD151 mRNA expression and as a result downregulates CD151 cell surface expression (FIG. 4E). Furthermore, blockage of CD151 activity (with a CD151 blocking antibody) results in decreased polymerization of actin filaments (FIG. 2E), inhibition of T cells migration in vitro (FIG. 2F) and their entry to the lymph nodes in vivo (FIG. 2H). Taken together, the present teachings indicate the major anti-inflammatory value of downregulating the CD151 pathway in lymphocytes, especially in T cells.

Thus, according to one aspect of the present invention there is provided a method of treating an inflammation in a subject in need thereof, the method comprising administering to the subject an agent capable of downregulating an activity or expression of CD151 in a lymphocyte, with the proviso that the agent is not CCL2, thereby treating the inflammation.

As used herein the term “subject in need thereof” refers to a mammal, preferably a human subject at any age which may benefit from the treatment modality of the present invention. According to specific embodiments, the subject does not suffer from a medical condition which is not associated with inflammation.

As used herein the term “treating” refers to preventing, curing, reversing, attenuating, alleviating, minimizing, suppressing or halting the deleterious effects of inflammation.

As used herein the term “inflammation” refers to any medical condition which comprises an inflammatory response in which migration of cells (e.g. to the lymph nodes) contributes to inflammation onset or progression.

A number of diseases and conditions, which involve an inflammatory response, can be treated using the methodology described hereinabove. Examples of such diseases and conditions are summarized infra.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Dickman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med. Sci. 2000 April; 319 (4):234), myasthenic diseases, Lambert-Eaton myasthenic syndrome (Takamori M. Am J Med. Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes. Examples of helper T lymphocyte-mediated hypersensitivity include, but are not limited to, Tₕ 1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J. Cardiol. 1999 Jun. 17; 83 (12A):75H), thrombocytopenic purpura (Moccia F. Ann Ital Med. Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Dickman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau YE. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns MP. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg AJ. J Clin Neurosci. 2000 May; 7 (3):191); Guillain-Barre syndrome and autoimmune neuropathies (Kusunoki S. Am J Med. Sci. 2000 April; 319 (4):234), myasthenia, Lambert-Eaton myasthenic syndrome (Takamori M. Am J Med. Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad. Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo Ti. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyper-acute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphocytic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

In a specific embodiment of the present invention, the inflammatory condition is a leukemia or lymphoma.

In another specific embodiment, the inflammatory condition is rheumatoid arthritis, inflammatory bowel disease (e.g. ulcerative colitis and Crohn's disease) and asthma.

As mentioned hereinabove, the method of this aspect of the present invention is affected by administering to the subject an agent capable of downregulating an activity or expression of CD151 in a lymphocyte to thereby treat the inflammation.

As used herein the term “lymphocyte” refers to a white blood cell of the immune system. A lymphocyte may include a T cell, a B cell and natural killer (NK) cell. According to a specific embodiment of the present invention, the lymphocyte is a T cell including CD4+ T cells, CD8+ T cells and any subsets therefrom (including naïve, mature and memory T cells).

As used herein the term “CD151” refers to a CD151 gene product (i.e., protein or mRNA) such as set forth in GenBank Accession Nos. NM_{—}004357.4, NM_{—}139030.3, NM_{—}139029.1, NM_{—}001039490.1, NP_{—}004348.2, NP_{—}620599.1, NP_{—}620598.1 and NP_{—}001034579.1.

As used herein “an agent capable of downregulating activity or expression of CD151” refers to a molecule that downregulates an activity or expression of CD151 per se (i.e., direct inhibition) or a down-stream signaling effector (i.e., indirect inhibition) thereof.

As used herein “CD151 activity” refers to a cell signaling activity (e.g., vav, β-catenin signaling), self-ligation activity (i.e., cd 151 dimerization), chemokine binding activity (e.g. CCL2), actin cytoskeleton polymerization, cell adhesion, cell spreading, cell migration (e.g. towards a chemokine such as CCL21 or CXCL12) or homing (e.g. to a lymphoid organ).

As used herein “CD151 expression” refers to expression of the CD151 membrane protein either at the protein level or at the mRNA level. Typically, CD151 is expressed on T cells, such as without limitation, naïve T cells, T helper cells, T-cytotoxic cells and NKT cells.

A number of agents can be used in accordance with this aspect of the present invention to downregulate an activity or expression of CD151 in a lymphocyte. Assays for qualifying such agents will be apparent to those of skill in the art. Thus, any in vitro or in vivo activity assay (e.g., signaling assay, reduction in cytoskeleton polymerization, reduction in cell migration) or dimerization assay (e.g. photobleaching, cross linking using radiolabeled ligand) can be used such as described in the Examples section which follows.

Downregulation of CD151 expression or activity can be effected on the genomic and/or the transcript level using a variety of molecules which interfere with transcription and/or translation (e.g., RNA silencing agents, Ribozyme, DNAzyme and antisense), or on the protein level using e.g., antagonists, enzymes that cleave the polypeptide, peptides which interfere with the self-ligation and the like. As mentioned above, downregulation of CD151 may be direct (i.e., on the mRNA or protein product of CD151 gene) or may be indirect such as by downregulating a CD151 effector (e.g. a downstream signaling molecule in the CD151 pathway, such as vav or β-catenin, or a cellular protein associated with CD151e.g. an integrin such as VLA4)

Following is a list of agents capable of downregulating expression level and/or activity of CD151.

One example, of an agent capable of downregulating a CD151 is an antibody or antibody fragment capable of specifically binding CD151. Preferably, the antibody specifically binds at least one extracellular epitope of a CD151 (e.g., amino acid coordinates 40-58 and 111-221 of human CD151).

According to some embodiments of the invention, the CD151 blocking antibody targets the peptide having an amino acid sequence set forth in SEQ ID NO: 129.

As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

According to one embodiment, the antibody is prepared using an antigenic peptide—for example having a sequence as set forth in SEQ ID NO: 129.

Generating antibodies to small peptides (e.g. peptides below a molecular weight of approximately 4000 daltons) may be carried out utilizing various methods know in the art (i.e. in order to overcome their weak immunogenic stimuli). Thus, for example, an animal (e.g. rabbit) may be immunized with the peptide of interest (e.g. the peptide as set in SEQ ID NO: 129) conjugated to a carrier molecule (for a general review of current methodology, see e.g. Van Regenmortel, et al., Synthetic Polypeptides as Antigens, Elsevier, 1988). Alternatively, antibodies to small peptides may be obtained by immunizing an animal (e.g. rabbit) with peptide-carrier conjugates, either with or without an adjuvant. After a series of immunization boosts, antibody-producing lymphocytes can be harvested and fused with myeloma cells. The fused cells may be grown out under conditions which select for stable hybridomas (described in detail in PCT Publication No. WO/1991/017177).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Since the antibody is capable of inhibiting CD151 ligation it may be referred to as a blocking antibody or a neutralizing antibody. An example of a commercially available antibody which can be used in accordance with the present teachings is the anti-CD151 antibody available from BD pharmingen. This blocking anti-CD151 antibody (BD pharmingen) has been described in detail in the Examples section below and in the literature [Sincock et al., J Cell Sci (1999) 112: 833-44; Shiomi et al., Lab Invest (2005) 85(12): 1489-506].

In another specific embodiment of the present invention, the antibody is directed to an effector of CD151 such as VLA4 or LFA1. Any VLA4 blocking antibody may be used in accordance with the present invention, as for example, anti-VLA4 antibody (e.g. available from Tocris Bioscience/Abcam). Furthermore, any LFA1 blocking antibody may be used in accordance with the present invention, as for example, anti-LFA1 antibody (e.g. available from R&D Systems). It will be appreciated that the antibody may be formulated to enter the cell. Methods of importing polypeptide agents to cells are well known in the art e.g., NLS-containing cell penetrating peptides, Zhang et al. PNAS (1998) 95(16):9184-9189 and WO 00/78346, each of which is hereby incorporated by reference in its entirety.

As mentioned, another agent capable of downregulating an activity or expression of CD151 or a downstream effector thereof is a nucleic acid agent suitable for silencing expression in a targeted manner. Examples of such agents are listed infra.

Downregulation of CD151 can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to down-regulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl. Acad. Sci. USA. 2002; 99:1443-1448; Tran N., et al., FEBS Lett. 2004; 573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA”. The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

Synthesis of RNA silencing agents suitable for use with the present invention can be effected as follows. First, the CD151 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

mRNAs to be targeted using RNA silencing agents include, but are not limited to, those whose expression is correlated with an undesired phenotypic trait. Exemplary mRNAs that may be targeted are those that encode truncated proteins i.e. comprise deletions. Accordingly the RNA silencing agent of the present invention may be targeted to a bridging region on either side of the deletion. Introduction of such RNA silencing agents into a cell would cause a down-regulation of the mutated protein while leaving the non-mutated protein unaffected.

For example, a suitable CD151 siRNA can be obtained from Santa Cruz Biotechnology (catalog number sc-42829). A suitable siRNA for an effector of CD151 may be, for example, an siRNA for vav (Santa Cruz Biotechnology, catalog number sc-29517) or an siRNA for VLA4 (Santa Cruz Biotechnology, catalog number sc-35685).

Another agent capable of downregulating a CD151 is a DNAzyme molecule capable of specifically cleaving an mRNA transcript or DNA sequence of the CD151. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a CD151 can also be effected by using an antisense polynucleotide capable of specifically hybridizing with an mRNA transcript encoding the CD151.

Design of antisense molecules which can be used to efficiently downregulate a CD151 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

For example, a suitable antisense oligonucleotides targeted against the CD151 mRNA (which is coding for the CD151 protein) would be SEQ ID NO: 6.

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a CD151 is a ribozyme molecule capable of specifically cleaving an mRNA transcript encoding a CD151. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

It will be appreciated that a non-functional analogue of CD151 can also be used as an agent which downregulates CD151 activity. An exemplary non-functional analogue which may be used according to the present teachings comprises a peptide having an amino acid sequence (typically no more than 150 amino acids in length) of a segment of the CD151 extracellular domain. The generated peptide specifically binds the extracellular domain of CD151 but does not enable CD151 dimerization (i.e. self-ligation) and activation thereof (e.g. signal transduction, cytoskeletal rearrangement, cell migration), such as by a competition mechanism. Examples of such peptides include SEQ ID NO: 7 and SEQ ID NOs: 11-112 which interact with a domain located in the region of amino acids 111-221 of human CD151 (SEQ ID NO: 5), SEQ ID NO: 9 and SEQ ID NOs: 113-122 which interact with a domain located in the region of amino acids 40-58 of human CD151 and SEQ ID NO: 129 which interact with a domain located in the region of amino acids 111-121 of human CD151.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom. These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of the present invention are preferably utilized in a linear form, although it will be appreciated that in cases where cyclicization does not severely interfere with peptide characteristics, cyclic forms of the peptide can also be utilized.

The peptides of the present invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of the present invention involves solid phase peptide synthesis.

Large scale peptide synthesis is described by Andersson Biopolymers 2000; 55(3):227-50.

It will be appreciated that each of the downregulating agents described hereinabove can be administered to the subject per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent capable of down-regulating an activity or expression of CD151 accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or to alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (an agent capable of downregulating an activity or expression of CD151) effective to prevent, alleviate or ameliorate symptoms of a disorder (e.g., inflammation) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Animal models for inflammatory colon diseases include animal models of ulcerative colitis such as, but are not limited to, trinitrobenzene sulfonic acid (TNBS)-induced colitis in rats and mice [Komori et al., J Gastroenterol (2005) 40: 591-599]. An animal model for adjuvant arthritis (AA, a model of rheumatoid arthritis) includes the rat heat-killed Mt strain H37Ra-induced AA [Kannan, Theor Biol Med. Model. (2005) 2:17]. An animal model for asthma includes the Ovalbumin (OVA) sensitization mouse model [Flaishon, L., et al., J. Immunol: Cutting edge 168: 3707 (2002)].

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide sufficient levels of the active ingredient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. The dosage and timing of administration will be responsive to a careful and continuous monitoring of the individual changing condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It will be appreciated that nucleic acid agents (as described above) can be administered to the subject employing any suitable mode of administration, described hereinbelow (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy). Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Depending on the medical condition, the subject may be administered with additional chemical drugs (e.g., anti-inflammatory).

Anti-inflammatory agents which may be used according to the present teachings include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and the include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Picomolar Levels of CCL2 Downregulate CD151 Expression

Materials and Experimental Procedures

Animals

C57BL/6 or Balb/c CCR2 deficient (CCR2−/−) mice were used at 6-8 weeks of age. All animal procedures were approved by the Animal Research Committee at the Weizmann Institute.

Cells

Spleen cells were obtained from mice as was previously described [Shachar and Flavell, Science (1996) 274: 106-108]. Spleen cells were incubated with anti-CD45R magnetic beads (B220; BD biosciences) and CD45 negative cells were collected for use.

Incubation of T Cells

1×10⁷ T cells from control C57BL/6 or CCR2−/− mice were suspended in 1 ml RPMI medium containing 10% (v/v) FCS. Next, 1 ng/ml of CCL2 (PeproTech) was added to half of the tubes (the other half was kept as a control), and the tubes were placed at 37° C. for different time periods (see the results section below). After incubation, the cells were washed and used for the different experiments.

Cytoskeleton Rearrangement

T cells were pre-incubated in the presence or absence of CCL2 (1 ng/ml, PeproTech) for 1 hour and were then stimulated with CCL21 (SLC, 100 ng/ml, PeproTech). Cells were fixed by 3.7% formaldehyde solution (Merck) and cytoskeleton rearrangement was analyzed by flow cytometry following staining with fluorescein isothiocyanate (FITC)-phalloidin, as was previously described [Flaishon et al., J. Biol. Chem. (2001) 276: 46701-46706].

RNA Isolation and Reverse Transcription

Total RNA was isolated from cells using the Tri reagent kit (Molecular Research Center, Inc). Reverse transcription was carried out using Superscript II RT (Gibco-BRL) and the primers:

Primers for HPRT:
(SEQ ID NO: 1)
5′ GAGGGTAGGCTGGCCTATGGCT 3′;
(SEQ ID NO: 2)
5′ GTTGGATACAGGCCAGACTTTGTTG 3′
Primers for CD 151:
(SEQ ID NO: 3)
5′ AGTACAAGCGTAACAGGTTC 3′;
(SEQ ID NO: 4)
5′ TTTCAGTGAGAGACGGGA 3′

Genechip

To obtain differential expression data of the T cells following CCL2 incubation, the differences between mRNA levels in this cell population were identified by screening mouse DNA chip arrays using the Affymatrix genechip expression analysis system (mouse 430 A2.0). The system consisted of two parameters: probe array and RNA target. Probe array included approximately 20,000 murine genes and ESTs. Each gene or EST was analyzed with approximately 20 pairs of specific, unique 25 mer-oligonucleotides probe. Each probe type was located in a specific area called probe cell. Each probe cell contained millions of copies of a given probe. All probe cells together made up the probe array (Affymetrix Genechip Expression Technical Manual). RNA from CCL2 treated T cells and from control T cells was converted to cDNA and then in vitro transcribed while labeled with biotin (see methods for RNA isolation and cells separation). The target RNA was hybridized to the probe array. The hybridized probe array was stained with streptavidin phycoerythrin conjugate and scanned by the GeneArray scanner. The amount of light emitted was proportional to the bound target at each location on the probe array. The two populations were compared according to the hybridization intensity in each cell.

Flow Cytometry

Freshly isolated T cells were pre-incubated in the presence or absence of CCL2 (1 ng/ml, PeproTech) for several time periods (see the results section below). After incubation the cells were double stained with anti-CD3 (Pharmagene) and anti-CD151 (Santa Cruz) and the secondary antibody alexa goat anti rabbit (Invitrogene) was used. Expression of the stained proteins was analyzed by flow cytometry.

Real-Time Reverse Transcription—PCR Analysis

mRNA levels of actin and CD151 were analyzed by quantitative real-time RT-PCR using a Light-Cycler instrument (Roche Diagnostics, Mannheim, Germany). The reaction volume (10 ml) contained 3 mM MgC12, LightCycler HotStart DNA SYBR Green I mix (Roche Diagnostics), specific primer pairs and 2.5 ml of cDNA.

PCR conditions were as follows:

10 minutes at 95° C. followed by 40-55 cycles of 15 seconds at 95° C., 15 seconds at 60° C., and 15 seconds at 72° C. PCR was performed in duplicates as previously described [Luger D et al. (2004) J Clin Immunol 24, 579-590]

Primer sequences were as follows:

Actin:
(SEQ ID NO: 123)
5-CAGTAACAGTCCGCCT-3
(SEQ ID NO: 124)
5-GTGACGTTGACATCCG-3
CD151:
(SEQ ID NO: 125)
5-TAAAGTGGAGGGTGGCTGTATCA-3
(SEQ ID NO: 126)
5-AGGTGAAGATCATGCCGAAGAC-3

Actin levels were used to normalize samples for calculation of the relative expression levels of the CD151 gene.

Results

As was previously reported [Flaishon et al., Blood (2008) 112: 5016-5025; PCT Publication No. WO 2008/012796], picomolar [pM] levels of CCL2 inhibit T lymphocyte homing to peripheral lymph nodes. Impaired homing of T lymphocytes to the peripheral lymph nodes results in attenuated progression of both asthma and adjuvant arthritis. Thus, low levels of circulating CCL2 can exert global suppressive effects on T-cell trafficking and differentiation within peripheral lymph nodes, and may be clinically beneficial as an anti-inflammatory agent.

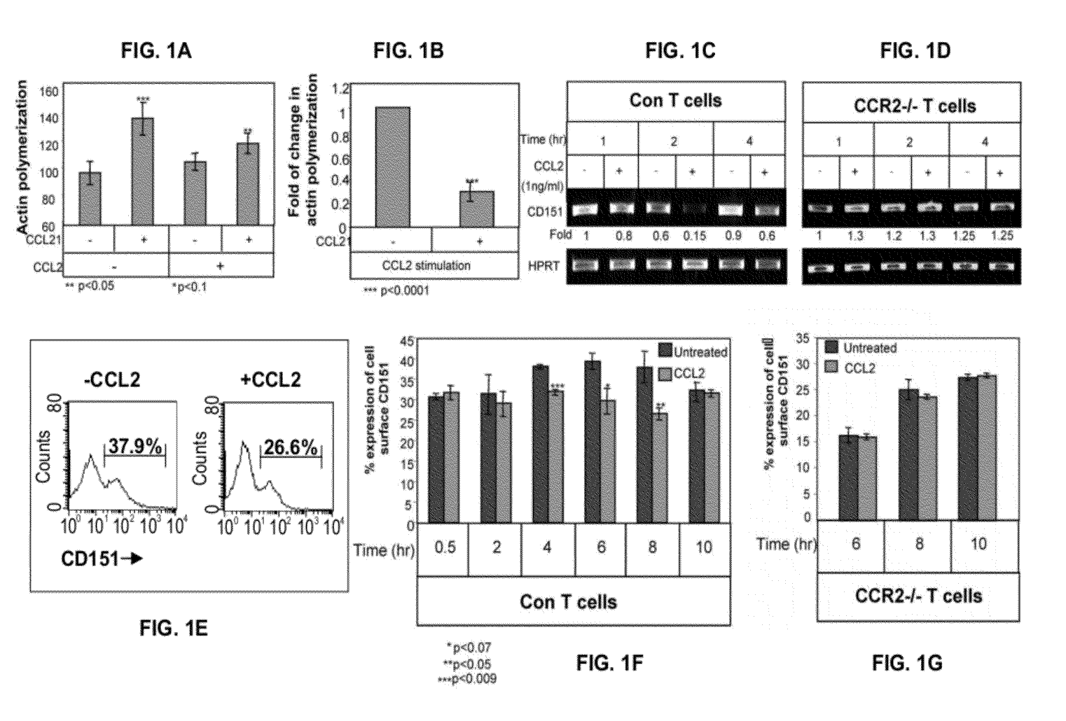

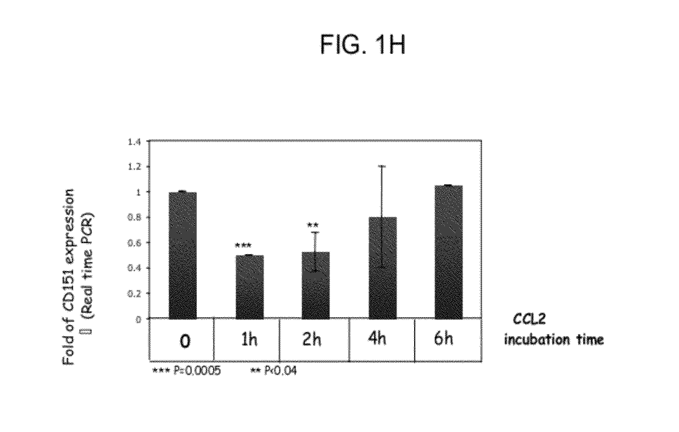

In order to uncover genes which are regulated by CCL2, T cells were incubated in the presence or absence of CCL2. To verify that indeed CCL2 had an effect on these cells, T cells were analyzed for their ability to polymerize actin after they were incubated for 1 hour with CCL2 and then stimulated with CCL21. As shown in FIGS. 1A-B, stimulation with CCL21 induced polymerization of actin in T cells whereas incubation with pM levels of CCL2 suppressed actin polymerization in these cells.

Next, T cells were incubated for 2 hours with CCL2 and their RNA was purified and analyzed for differential display of genes using DNA chip array. One of the genes that were found to be downregulated by CCL2 was CD151 (FIGS. 1C-D).

In order to verify the genechip results, T cells were pre-incubated in the presence or absence of CCL2 for 1, 2 or 4 hours, and RNA from each group was purified. RT-PCR was carried out and the transcription of CD151 was analyzed. As is illustrated in FIG. 1C, CD151 expression was clear in control naïve T cells and was downregulated following 2 and 4 hour incubations with pM levels of CCL2. This effect was specific to CCL2-CCR2 interaction, as no inhibition was observed in CCR2-deficient cells (CCR2−/− T cells, FIG. 1D). The results were further collaborated using Real time PCR using primers for C151 and actin (see materials and experimental procedures section above, FIG. 1H). In order to determine whether CD151 cell surface expression was downregulated following CCL2 incubation, control and CCR2−/− T cells were incubated in the presence or absence of CCL2 for different time periods and CD151 expression was determined. As demonstrated in FIGS. 1E-F, CCL2 downregulated CD151 cell surface expression levels, while no change was detected in CCR2−/− T cells (FIG. 1G).

### Example 2

### CD151 Regulates T Cells Cytoskeleton Rearrangement and Migration

Materials and Experimental Procedures

Animals

As depicted in detail in Example 1, above.

Cells

As depicted in detail in Example 1, above.

CD151 Activation

T cells were pretreated with either 2 μg/ml of activating antibody specific for the extracellular domain of CD151 (Santa Cruz) or with an isotype control antibody IgG rabbit anti mouse (ImmunoResearch Labs.inc, 2 μg/ml)/anti CUL1 (Santa Cruz) for 1 hour/20 hours.

RNA Isolation and Reverse Transcription

As depicted in detail in Example 1, above.

Cytoskeleton Rearrangement

For CD151 activation leading to cytoskeletal rearrangement-T cells were pretreated with either 2 μg/ml of activating antibody specific for the extracellular domain of CD151 (Santa Cruz) or with an isotype control antibody IgG rabbit anti mouse (ImmunoResearch Labs.inc)/anti CUL1 (Santa Cruz) for 1 hour/20 hours.

For blocking CD151− T cells were pretreated for 1 hour with 10 μg/ml blocking anti-CD151 antibody (BD pharmingen) or with 10 μg/ml control antibody anti-IgG (ImmunoResearch Labs.inc)/anti KPL1 (Chemicon).

Next, a 100 ng/ml chemokine CCL21 (SLC, PeproTech) stimulation was carried out and the cells were fixed by 3.7% formaldehyde solution (Merck), permeabilized and stained with fluorescein isothiocyanate (FITC)-phalloidin, as was previously described [Flaishon et al., J. Biol. Chem. (2001) 276: 46701-46706]. Cytoskeleton rearrangement was analyzed by flow cytometry.

Transwell Migration

Chemotaxis was assayed using transwell chambers as was previously described [Flaishon et al. (2001), supra]. Briefly, about 4×10⁶ T cells were pretreated with different antibodies (see methods of cytoskeleton rearrangement, above) for 1 hour. The migration towards the chemokine CCL21 (SLC, 0.4 mg/ml, PeproTech), residing in the lower part of the apparatus, through wells coated or uncoated with fibronectin, was analyzed after 3 hours by FACSort.

Tracking of Cells In Vivo

Naive T cells were stained with 5 μM fluorescent dye carboxyfluorescein diacetate succinimidyl ester (CFDA-SE; Invitrogen, Carlsbad, Calif.) for 15 minutes. Next, the cells were preincubated with either the blocking antibody anti-CD151 or with an isotype control antibody (10 μg/ml) for 1 hour. The homing of T cells was analyzed as was previously described [Carlos and Harlan, Blood (1994) 84(7): 2068-101]. In short, the cells were injected intravenously into C57BL/6 mice and the peripheral lymph nodes (PLNs) were collected after 3 hours. The proportion of labeled cells (FITC-positive population) recovered from PLNs was determined compared to the control population (treated with anti IgG).

Results

It was previously shown in human melanoma cells that homophilic interactions between CD151 proteins on the surface of neighboring cells lead to a signaling cascade that results in enhanced cell motility [Hong et al., J Biol Chem (2006) 281(34): 24279-92]. Therefore, in order to determine whether CD151 regulates T cell migration and homing, the inventors of the present invention activated CD151 using an anti-CD151 antibody which mimics the CD151 homophilic interactions. This anti-CD151 antibody binds the extracellular loop of CD151 tetraspanin and transmits a signal that results in activation of downstream cascades.

To first check whether activation with anti-CD151 antibody autoregulates its own expression, T cells were incubated with either the activating anti-CD151 antibody or with an isotype control IgG antibody for several time periods and CD151 mRNA levels were analyzed. As can be seen in FIG. 2A, CD151 activation elevated CD151 mRNA levels, thus autoregulated its own expression. After 4 and 6 hours there was a dramatic elevation in CD151 gene expression.

Among the requirements for inducible integrin-mediated adhesion are an increased rate of actin polymerization and an extensive reorganization of the actin-based cytoskeleton. Chemokines promote a rapid burst of actin polymerization, which peaks at 30 seconds to 1 minute and subsides to basal levels within 5 to 10 minutes post-stimulation [Samstag et al., J Leukoc Biol (2003) 73(1): 30-48]. Therefore, in order to determine whether CD151 plays a role in T cell migration, cytoskeleton rearrangement of T cells was evaluated after cells were stimulated with the activating anti-CD151 or isotype control antibodies. As shown in FIG. 2B, CD151 activation augmented the cells' ability to polymerize their actin (around 50% increase) as compared to the effect of the control antibody.

Next, the role of CD151 in T cells migration in vitro was examined using a transwell migration assay. T cells were pretreated with either anti-CD151 or with an isotype control antibody for 1 hour. Migration towards the chemokine CCL21 (SLC) residing in the lower part of the apparatus was analyzed after 3 hours by FACSort. As shown in FIG. 2C, CD151 activation dramatically elevated the migration of T cells towards CCL21 as compared to the control antibody.

T cell migration across tissue barriers and through the extracellular matrix (ECM) is a key event during inflammation that is mediated primarily by β1-integrins, which function as cell surface receptors for ECM proteins such as fibronectin (FN) [Steffen et al., Am J Pathol (1996) 148(6): 1819-38]. To determine whether CD151 regulates migration across the ECM, T cell migration was analyzed through fibronectin (FN)-coated wells. T cells were pretreated with anti-CD151 or an isotype control antibody for 1 hour and migration towards the chemokine CCL21 (SLC) residing in the lower part of the apparatus through FN-coated wells was analyzed after 3 hours by FACSort. As shown in FIG. 2D, activation of CD151 significantly elevated T cell migration through FN-coated wells towards CCL21 as compared to the control antibody. Thus, CD151 activation induced T cell cytoskeleton rearrangement and migration (across the ECM) in a CCL21 dependent manner.

The effect of CD151 inhibition on T cell migration was evaluated next. T cells were pretreated for 1 hour with a CD151 blocking antibody, that was previously shown to block the migration of endothelial cells [Sincock et al., J Cell Sci, (1999) 112 (Pt 6): 833-44] or with a control antibody (10 μg/ml). Cells were then stimulated with CCL21, fixed, permeabilized, stained with FITC-phalloidin and analyzed by flow cytometry. As can be seen in FIG. 2E, CD151 blocking antibody reduced, by about 40%, the CCL21 induced actin polymerization. In addition, blocking CD151 dramatically reduced the CCL21 induced transwell migration compared to the control antibody (FIG. 2F). However, no inhibition was recorded for T cell migration through FN-coated wells (FIG. 2G). It is possible that the high migration percentage through the FN-coated wells overcame the effectiveness of the inhibition of the blocking anti-CD151 antibody. Alternatively, it is possible that an allosteric binding between CD151 and the FN receptor, the β1 integrin, may prevent the binding between the blocking anti-CD151 antibody and the CD151 on the T cell surface.

The inhibitory effect of the blocking antibody on in vitro chemotaxis of T lymphocytes suggested that this blockage could also interfere with the homing of naive T cells into LNs, a process critically dependent on CCL21-triggered adhesion and motility. Next, the inventors analyzed the ability of anti-CD151-treated naive T cells to enter into peripheral LNs (PLNs). As shown in FIG. 2H, the proportion of anti-CD151 treated T cells recovered from PLNs of C57BL/6 mice 3 hours after injection was much lower compared with the control population (treated with anti IgG), thus, there was a significant reduction in the homing of anti-CD151-treated naive T cells into the LN. These results clearly demonstrate that blockage of CD151 inhibited the migration of naive T cells into LNs in vivo.

### Example 3

### Interaction of CD151 with Integrins Induces a Signaling Cascade in T Cells

Materials and Experimental Procedures

Cells

As depicted in detail in Example 1, above.

CD151 Stimulation

1×10⁷ T cells were suspended in 1 ml RPMI medium containing 10% (v/v) FCS. Next, the cells were stimulated with either 2 μg/ml anti-CD151 (Santa Cruz) or an isotype control antibody IgG rabbit anti mouse (ImmunoResearch Labs.inc, 2 μg/ml). The tubes were immediately placed at 37° C. for several time periods (see the results section below). Immediately after stimulation, the cells were washed and taken for the experiments. For preparation of cell extract, the cells were fast frozen in liquid Nz.

Preparation of Cell Extract

Stimulated frozen cells were lysed in lysis buffer containing: 25 mM Tris, pH 7.4; 2 mM Vanadate; 75 mM glycophosphate, pH 7.2; 2 mM EDTA; 2 mM EGTA; 10 mM NaPPi; and 0.5% NP-40, in the presence of the following protease inhibitors: 10 mg/ml Leupeptin; 10 mg/ml aprotinin; 10 mg/ml pepstatin; 10 mg/ml chymostatin (Roche); 1 mM PMSF (Sigma); and 20 mM N-etheyl-melamide (Sigma).

ERK Detection

Detection of ERK phosphorylation was performed as was previously described [Flaishon et al., Blood (2004) 104: 933-941].

Immunoprecipitation

Protein G-Sepharose beads (GE Healthcare) were conjugated to Tyr (P) monoclonal antibody (Santa Cruz) or to anti-CD151 (Santa Cruz) for 2 hours at 4° C. followed by three washes in phosphate-buffered saline. Beads were added to the cell lysates and proteins were immunoprecipitated overnight. The protein G-bound material was washed three times with phosphate-buffered saline containing 0.1% SDS and 0.5% Nonidet P-40. Immunoprecipitates were separated by SDS-PAGE. The protein bands were transferred onto a nitrocellulose membrane and probed with anti-Vav (UBI, Hauppauge, N.Y.), anti β-cat (BD Transduction Laboratories) or anti-β1 integrin (Santa Cruz) followed by horseradish peroxidase-conjugated anti-mouse or rabbit IgG (Jackson ImmunoResearch Labs, West Grove, Pa.).

Immunoprecipitation of LFA1

Protein G-Sepharose beads (GE Healthcare) were conjugated to either 2 μg/ml of anti-CD151 (Santa Cruz) or an isotype control antibody IgG rabbit anti mouse (ImmunoResearch Labs.inc) for 2 hours at 4° C. followed by three washes in phosphate-buffered saline. Beads were added to the cell lysates and proteins were immunoprecipitated overnight. The protein G-bound material was washed three times with phosphate-buffered saline containing 0.1% SDS and 0.5% Nonidet P-40. Immunoprecipitates were separated by SDS-PAGE. The protein bands were transferred onto a nitrocellulose membrane and probed with anti-aL integrin (Santa Cruz) followed by horseradish peroxidase-conjugated rabbit IgG (Jackson ImmunoResearch Labs, West Grove, Pa.).

Detection of β-Catenin Phosphorylation

1×10⁷ T cells were suspended in 1 ml RPMI medium containing 10% (v/v) FCS. The cells were stimulated with either 2 μg/ml anti-CD151 (Santa Cruz) or an isotype control antibody IgG rabbit anti mouse (ImmunoResearch Labs.inc). Thereafter, the cells were immediately placed at 37° C. for 5 minutes. Immediately after stimulation, the cells were fast frozen in liquid N2 and were lysed in lysis buffer containing: 25 mM Tris, pH 7.4; 2 mM Vanadate; 75 mM glycophosphate, pH 7.2; 2 mM EDTA; 2 mM EGTA; 10 mM NaPPi; and 0.5% NP-40, in the presence of the following protease inhibitors: 10 mg/ml Leupeptin; 10 mg/ml aprotinin; 10 mg/ml pepstatin; 10 mg/ml chymostatin (Roche); 1 mM PMSF (Sigma); and 20 mM N-etheyl-melamide (Sigma).

Protein G-Sepharose beads (GE Healthcare) were conjugated to Tyr (P) monoclonal antibody (Santa Cruz) for 2 hours at 4° C., followed by three washes in phosphate-buffered saline. Beads were added to the cell lysates and proteins were immunoprecipitated overnight. The protein G-bound material was washed three times with phosphate-buffered saline containing 0.1% SDS and 0.5% Nonidet P-40. Immunoprecipitates were separated by SDS-PAGE. The protein bands were transferred onto a nitrocellulose membrane and probed with anti β-cat (BD Transduction Laboratories) followed by horseradish peroxidase-conjugated anti-mouse IgG (Jackson ImmunoResearch Labs, West Grove, Pa.).

Detection of Vav Phosphorylation

T cells were pretreated for several time periods with 10 μg/ml blocking anti-CD151 antibody (BD pharmingen) or with a control antibody anti-IgG (ImmunoResearch Labs.inc). The cells were then fast frozen in liquid N2 and were lysed in lysis buffer containing: 25 mM Tris, pH 7.4; 2 mM Vanadate; 75 mM glycophosphate, pH 7.2; 2 mM EDTA; 2 mM EGTA; 10 mM NaPPi; and 0.5% NP-40, in the presence of the following protease inhibitors: 10 mg/ml Leupeptin; 10 mg/ml aprotinin; 10 mg/ml pepstatin; 10 mg/ml chymostatin (Roche); 1 mM PMSF (Sigma); and 20 mM N-etheyl-melamide (Sigma).

Protein G-Sepharose beads (GE Healthcare) were conjugated to Tyr (P) monoclonal antibody (Santa Cruz) for 2 hours 4° C., followed by three washes in phosphate-buffered saline. Beads were added to the cell lysates and proteins were immunoprecipitated overnight. The protein G-bound material was washed three times with phosphate-buffered saline containing 0.1% SDS and 0.5% Nonidet P-40. Immunoprecipitates were separated by SDS-PAGE. The protein bands were transferred onto a nitrocellulose membrane and probed with anti-Vav (UBI, Hauppauge, N.Y.) followed by horseradish peroxidase-conjugated anti-mouse IgG (Jackson ImmunoResearch Labs, West Grove, Pa.).

Results

It was previously shown that CD151 has a strong molecular association with the β1 family of integrins on different cell types, such as epithelial cells [Chattopadhyay et al., J Cell Biol (2003) 163(6): p. 1351-62]. Among beta 1 integrins in naïve T cells, VLA4 (β1α4) is the predominant integrin-expressed. Therefore, the inventors wished to examine whether CD151 forms a complex with β1-integrins on naïve T cells by co-immunopecipitation. T cells were lysed and CD151 was immunoprecipitated. Immunoprecipitates were separated on SDS-PAGE and analyzed by anti-β1 integrin antibody. As shown in FIG. 3A, CD151 specifically pulled down β1 integrin, showing that CD151 forms a complex with VLA4 in T cells. This interaction seemed to be stable, in the basal state of the cells, regardless to any exposure of cytokines.

In order to examine whether CD151 also forms a complex with LFA1 integrin on naïve T cells, a co-immuno precipitation was performed. Accordingly, T cells were lysed, CD151 was immunoprecipitated and the resultant immunoprecipitates were separated on SDS-PAGE and analyzed by anti-αL integrin antibody. As shown in FIG. 3B, CD151 specifically pulled down αL integrin, showing that CD151 forms a complex with LFA1 in T cells.

The CD151-induced signaling cascade was then examined. Inventors tested whether T cells migration-induced by CD151 activation also correlates with β-catenin phosphorylation. T cells were pretreated with either activating anti-CD151 or an isotype control antibody (2 μg/ml) for 5 and 10 minutes. The cells were then lysed and β-catenin phosphorylation was analyzed. As can be seen in FIG. 3C, CD151 activation induced phosphorylation of the β-catenin. Taken together, these results show that CD151 influence on the T cell migration and trafficking is mediated, at least in part, by wnt and β-catenin signaling.

It was previously shown that CCL2 mediated inhibition of actin polymerization motility and integrin adhesion strengthening involves interference with Vav1 function [Flaishon et al., Blood (2008) 112(13): 5016]. In order to determine whether CD151 induction of actin polymerization motility involves Vav1 activation, inventors analyzed Vav phosphorylation in cells stimulated with activating anti-CD151 or with an isotype control antibody for several time periods. As can be seen in FIG. 3D, CD151 stimulated Vav phosphorylation essential for its Guanine-nucleotide exchange factors (GEF) activities in order to influence the T cell migration. Inventors next analyzed whether Vav phosphorylation is influenced by treatment with 10 μg/ml blocking anti-CD151 antibody (BD pharmingen) or with a control antibody anti-IgG (ImmunoResearch Labs.inc). As is illustrated in FIG. 3F, blockage of CD151 leads to down-regulation of Vav phosphorylation.

It was previously shown that CCL2 downregulates a signaling cascade that involves ERK1/2, resulting in inhibition of cytoskeleton rearrangement and migration [Flaishon et al. (2008), supra]. To further evaluate the signaling cascade transmitted by CD151 activation, T cells were pretreated with either activating anti-CD151 or with an isotype control antibody for several time periods. The T cells were then lysed and ERK1/2 phosphorylation was analyzed. As can be seen in FIG. 3E, CD151 activation did not induce ERK phosphorylation. Therefore, the signal transduction transmitted by CD151 did not seem to involve ERK1/2.

### Example 4

### CCL2 Interferes with the Signals Transmitted by CD151

Materials and Experimental Procedures

Cells

As depicted in detail in Example 1, above.

Incubation of T Cells

As depicted in detail in Example 1, above.

Preparation of Cell Extract

As depicted in detail in Example 3, above.

Immunoprecipitation

As depicted in detail in Example 3, above.

CD151 Stimulation

1×10⁷ T cells were incubated in the presence or absence of CCL2 for 10 minutes, followed by stimulation with either activating anti-CD151 (Santa Cruz, 2 μg/ml) or an isotype control antibody IgG rabbit anti mouse (ImmunoResearch Labs.inc, 2 μg/ml) for 1 hour. The cells were then stimulated with CCL21 (100 ng/ml) and actin polymerization was analyzed.

Cytoskeleton Rearrangement

Cells were fixed by 3.7% formaldehyde solution (Merck), and cytoskeleton rearrangement was analyzed by flow cytometry following staining with fluorescein isothiocyanate (FITC)-phalloidin, as was previously described [Flaishon et al. (2001), supra].

Results

As the results showed that CD151 and VLA4 formed a complex on naïve T cells, the inventors sought next to determine whether pM levels of CCL2 inhibited T cell migration by dissociating the CD151/VLA4 complex.

T cells from control mice were incubated with or without CCL2 for several time periods. Cells were then lysed and CD151 was immunoprecipitated. Immunoprecipitates were separated on SDS-PAGE and analyzed by anti-β1 integrin antibody. As depicted in FIG. 4A, incubation with CCL2 resulted in the disappearance of the CD151/VLA4 complex, while it had no effect on CCR2−/− T cells (FIG. 4B). Thus, CCL2 induces a signal through CCR2 that results in the dissociation of the CD151/VLA4 complex.

To determine whether the complex dissociation interferes with CD151 activity, T cells were incubated in the presence or absence of CCL2 for 10 minutes, followed by stimulation with either activating anti-CD151 or an isotype control antibody for 1 hour. The cells were then stimulated with CCL21 and actin polymerization was analyzed. CD151 activation induced actin polymerization, an elevation which was completely abolished by pre-incubating the cells with pM levels of CCL2 (prior to activation of CD151, FIG. 4C). This effect was not apparent in cells lacking CCR2 (FIG. 4D). These results suggest that the CD151/VLA4 complex is essential for the ability of CD151 to induce a signaling cascade resulting in the ability of the cells to polymerize their actin. pM levels of CCL2 dissociates this complex by binding to its receptor which leads to inhibition of the CD151 induced cascade.

This effect was also maintained in longer CCL2-incubation periods (FIG. 4E). This effect may be due to the dissociation of the CD151/VLA4 complex by CCL2 or due to the lower levels of CD151 on the T cell surface following CCL2 incubation for longer time periods.

### Example 5

### CCL2 Interferes with CD151 Signaling Cascade by Inhibiting β-Catenin Phosphorylation

Materials and Experimental Procedures

CCL2 Stimulation and Detection of β-Catenin Phosphorylation

1×10⁷ T cells were incubated with 1 ng/ml CCL2 for 10 minutes. Next, the cells were stimulated with either 2 μg/ml anti-CD151 (Santa Cruz) or an isotype control antibody IgG rabbit anti mouse (ImmunoResearch Labs.inc) for 5 minutes. Immediately after stimulation, the cells were fast frozen in liquid N2 and were lysed in lysis buffer containing: 25 mM Tris, pH 7.4; 2 mM Vanadate; 75 mM glycophosphate, pH 7.2; 2 mM EDTA; 2 mM EGTA; 10 mM NaPPi; and 0.5% NP-40, in the presence of the following protease inhibitors: 10 mg/ml Leupeptin; 10 mg/ml aprotinin; 10 mg/ml pepstatin; 10 mg/ml chymostatin (Roche); 1 mM PMSF (Sigma); and 20 mM N-etheyl-melamide (Sigma).

Protein G-Sepharose beads (GE Healthcare) were conjugated to Tyr (P) monoclonal antibody (Santa Cruz) for 2 hours at 4° C., followed by three washes in phosphate-buffered saline. Beads were added to the cell lysates and proteins were immunoprecipitated overnight. The protein G-bound material was washed three times with phosphate-buffered saline containing 0.1% SDS and 0.5% Nonidet P-40. Immunoprecipitates were separated by SDS-PAGE. The protein bands were transferred onto a nitrocellulose membrane and probed with anti β-cat (BD Transduction Laboratories) followed by horseradish peroxidase-conjugated anti-mouse IgG (Jackson ImmunoResearch Labs, West Grove, Pa.).

Results

As shown above (see Example 4), CD151 activation induces β-catenin phosphorylation. In order to investigate whether CCL2 interferes with the CD151 signaling cascade the T cells were pre-incubated with CCL2 and then were stimulated with anti-CD151 activating antibody. As shown in FIG. 4F, β-catenin (β-cat) phosphorylation was completely abolished by pre-incubation with picomolar (pM) levels of CCL2 prior to activation of CD151. Taken together, these results illustrate that CD151/VLA4 complex is essential for of the ability of CD151 to induce a signaling cascade. Furthermore, CCL2 dissociation of this complex leads to inhibition of the CD151 signaling cascade.

### Example 6

### The effect of CD151 Inhibition and Activation on LFA-1 Mediated Adhesion to ICAM-1 Triggered by CCL21 and Adhesion Strengthening in Mouse T Cells

Materials and Experimental Procedures

Laminar Flow Adhesion Assays

Purified ICAM-1-Fc (R&D Systems, Minneapolis, Minn.) were coated alone or with mouse CCL21 (R&D Systems) on polystyrene plates, as previously described [Grabovsky V et al. (2000) J Exp Med. 192, 495-506; Sigal A et al. (2000) J. Immunol. 165, 442-452]. The polystyrene plates were each assembled on the lower wall of the flow chamber as previously described [Feigelson S W et al. (2001) J Biol. Chem. 276, 13891-13901]. All flow experiments were conducted at 37° C.

Mouse T cells were purified and re-suspended in HBSS++ (HBSS, Mg²⁺Ca²⁺) with ICAM-1-Fc or mCCL2 for 1 hr. Cells were then perfused through the flow chamber at low shear stress (0.5-0.75 dyn/cm2) for 2 min. Tethers were defined as transient or rolling if cells attached briefly (less than 2 seconds) to the substrate, and as arrests if cells immediately arrested and remained stationary for at least 5 seconds of continuous flow. To assess adhesion strengthening, post arrest resistance to detachment over time was determined. Accumulated cells were subjected to a shear stress of 5 dyn/cm² for 10 minutes. At the indicated time points, the number of cells that remained bound was expressed relative to T cell population originally accumulated in the first 2 minutes of the assay.

Results

Treatment of mouse T cells with 1 ng/ml mCCL2 had a minor effect on LFA-1 activation by surface bound CCL21, as evident from the normal capacity of CCL2 treated T cells to arrest (stick) on the LFA-1 ligand, ICAM-1. Nevertheless, once arrested on this ligand, CCL2 pretreated T cells failed to remain firmly adhered to the integrin ligand for prolonged periods and readily detached from the ligand when exposed to continuous application of shear stress for several minutes after initial sticking (FIGS. 4G-H).

Pre-treating mouse T cells with the CD151 inhibitor Ab (10 μg/ml) inhibited rapid LFA-1 activation triggered by mCCL21, as the cells failed to accumulate when applying low shear stress. The inhibition of CD151 didn't affect the ability of the cells to undergo adhesion straightening after cell arrest, as the cells were resisted to detachment when applying high shear stress in comparison to control (FIGS. 4I-J).

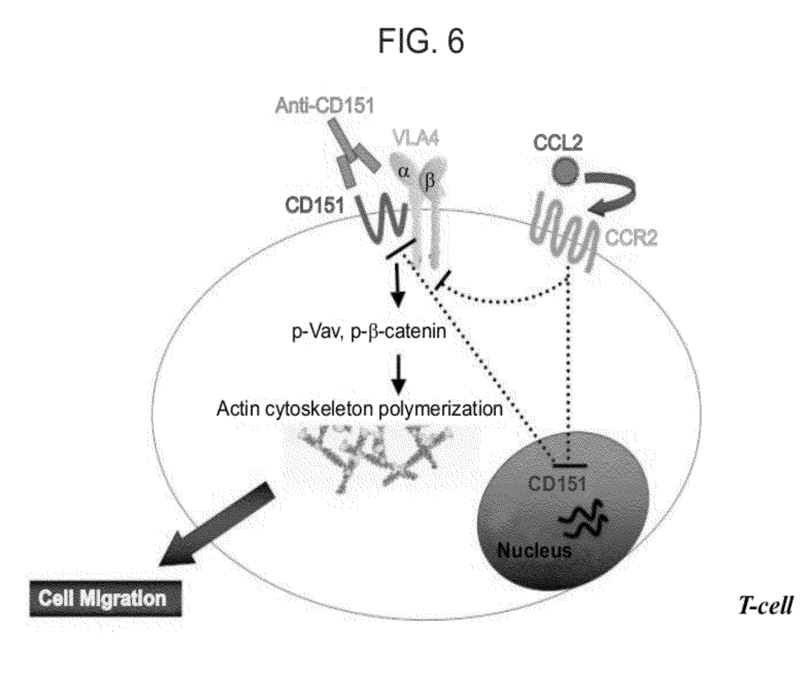

Taken together, the results of the present invention indicated a key role for CD151 in T cell migration and homing in a CCL2 dependent manner (FIG. 6). According to the present model, CD151 forms a complex with VLA4, the predominant integrin expressed among β1 integrins on naïve T cells, which initiates a signaling cascade that involves phosphorylation of β-catenin and Vav, leading to cytoskeleton rearrangement and migration of the cells. Blockage of CD151 activity resulted in decreased polymerization of actin filaments, inhibition of T cells migration in vitro and their entry to the LNs in vivo. pM levels of CCL2, which bind to the CCR2 receptor, had both short and long term effects on CD151. In the short term effect, CCL2 immediately dissociated the CD151/VLA4 complex, resulting in an immediate inhibition of CD151-induced signaling cascade and downregulation of actin cytoskeleton and migration. After a few hours, CCL2 downregulated CD151 mRNA expression and as a result downregulated CD151 cell surface expression.

### Example 7

### Construction of CD151 Constructs

Constructs

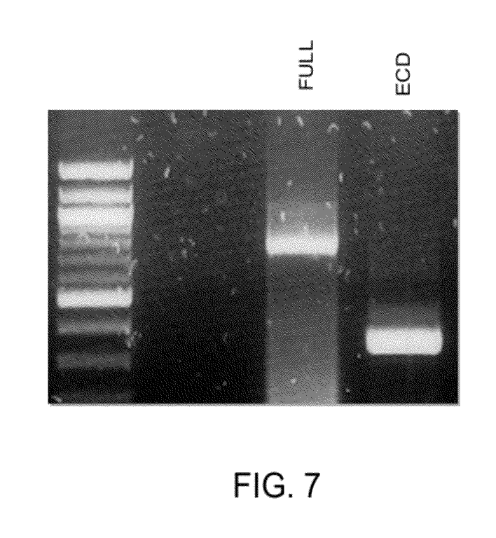

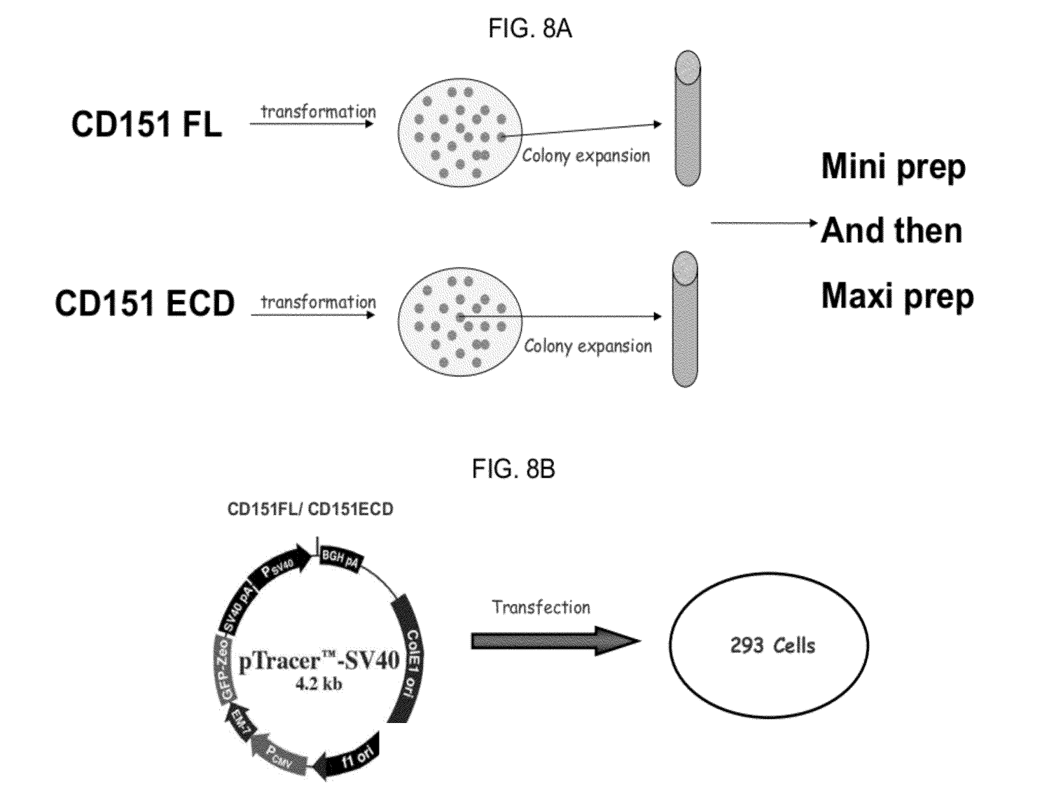

The full-length human CD151 (FL-CD151, SEQ ID NO: 127) and the extracellular domain human CD151 (ECD-CD151, SEQ ID NO: 128) constructs in the pEF4/Myc-His vector (Invitrogen) vector were designed and purified (FIGS. 7 and 8A-B). Next, the constructs (CD151 FL/ECD) were transformed into bacteria, the colonies were expanded and the plasmids purified (FIG. 8A). Finally, the plasmids containing the CD151 FL or CD151 ECD were transfected into 293 cells (FIG. 8B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating an inflammation in a subject in need thereof, the method comprising administering to the subject a peptide capable of downregulating an activity of CD151 in a lymphocyte, thereby treating the inflammation.

2-3. (canceled)

4. A pharmaceutical composition comprising as an active ingredient a peptide capable of downregulating an activity of CD151 in a lymphocyte, and a pharmaceutically acceptable carrier.

5-14. (canceled)

15. The method of claim 1, wherein the subject is a human subject.

16. The method of claim 1, wherein said inflammation is associated with a medical condition selected from the group consisting of a cancer, an autoimmune disease, a hypersensitivity, a diabetes, an infectious disease, a transplantation associated disease and an allergy.

17. The method of claim 1, wherein said lymphocyte is a T cell.

18. The method of claim 1, wherein said downregulating an activity of said CD151 inhibits lymphocyte migration or homing.

19. The method of claim 18, wherein said migration or homing is to a lymphoid organ.

20. (canceled)

21. A method of treating an inflammation in a subject in need thereof, the method comprising administering to the subject a CD151 blocking antibody capable of downregulating an activity of CD151 in a lymphocyte, with the proviso that said inflammation is not cancer, thereby treating the inflammation.

22. A method of inhibiting lymphocyte migration or homing in a subject in need thereof, the method comprising administering to the subject a peptide capable of downregulating an activity of CD151 in said lymphocyte, thereby inhibiting said lymphocyte migration or homing.

23. A method of inhibiting lymphocyte migration or homing in a subject in need thereof, the method comprising administering to the subject a CD151 blocking antibody capable of downregulating an activity of CD151 in said lymphocyte, thereby inhibiting said lymphocyte migration or homing.

24. A pharmaceutical composition comprising as an active ingredient a CD151 blocking antibody capable of downregulating an activity of CD151 in a lymphocyte and a pharmaceutically acceptable carrier.

25. The method of claim 1, wherein said peptide is a CD151 derived peptide capable of blocking dimerization of said CD151 or an interaction thereof with a downstream effector.

26. The method of claim 25, wherein said effector is selected from the group consisting of VLA4 or LFA1.

27. The method of claim 1, wherein said peptide interacts with a domain located in the region of amino acids 111-221 of human CD151.

28. The method of claim 27, wherein said peptide is as set forth in SEQ ID NO: 7 or SEQ ID NOs: 11-112.

29. The method of claim 1, wherein said peptide interacts with a domain located in the region of amino acids 40-58 of human CD151.

30. The method of claim 29, wherein said peptide is as set forth in SEQ ID NO: 9 or SEQ ID NOs: 113-122.

31. The method of claim 1, wherein said peptide interacts with a domain located in the region of amino acids 111-121 of human CD151.

32. The method of claim 31, wherein said peptide is as set forth in SEQ ID NO: 129.

33. The method of claim 21, wherein said CD151 blocking antibody is capable of binding at least one extracellular epitope of said CD151.

34. The method of claim 33, wherein said extracellular epitope of said CD151 comprises amino acid coordinates 40-58 of human CD151.

35. The method of claim 33, wherein said extracellular epitope of said CD151 comprises amino acid coordinates 111-221 of human CD151.

36. The method of claim 21, wherein said CD151 blocking antibody targets the peptide having an amino acid sequence set forth in SEQ ID NO: 129.

37. The method of claim 21, wherein said inflammation is associated with a medical condition selected from the group consisting of an autoimmune disease, a hypersensitivity, a diabetes, an infectious disease, a transplantation associated disease and an allergy.


SEQUENCE LISTING (129 sequences)

SEQ ID NO: 1 - DNA, 22 bp, Artificial sequence
  Single strand DNA oligonucleotide
gagggtaggctggcctatggct

SEQ ID NO: 2 - DNA, 25 bp, Artificial sequence
  Single strand DNA oligonucleotide
gttggatacaggccagactttgttg

SEQ ID NO: 3 - DNA, 20 bp, Artificial sequence
  Single strand DNA oligonucleotide
agtacaagcgtaacaggttc

SEQ ID NO: 4 - DNA, 18 bp, Artificial sequence
  Single strand DNA oligonucleotide
tttcagtgagagacggga

SEQ ID NO: 5 - protein, 253 aa, Homo sapiens
MGEFNEKKTTCGTVCLKYLLFTYNCCFWLAGLAVMAVGIWTLALKSDYISLLASGTYLATAYILVVAGTVVMVTGVLGCCATFKERRNLLRLYFILLLIIFLLEIIAGILAYAYYQQLNTELKENLKDTMTKRYHQPGHEAVTSAVDQLQQEFHCCGSNNSQDWRDSEWIRSQEAGGRVVPDSCCKTVVALCGQRDHASNIYKVEGGCITKLETFIQEHLRVIGAVGIGIACVQVFGMIFTCCLYRSLKLEHY

SEQ ID NO: 6 - DNA, 1572 bp, Homo sapiens
attctcagcgctgggagccgccgcccccgcagctgctgccgccgccgccagggcccggactcggacgcgtggtagcctagagtcctggggagcttctgtccacctgtcctgcagaggagtcgtttccagcccggctgccccaggatgggtgagttcaacgagaagaagacaacatgtggcaccgtttgcctcaagtacctgctgtttacctacaattgctgcttctggctggctggcctggctgtcatggcagtgggcatctggacgctggccctcaagagtgactacatcagcctgctggcctcaggcacctacctggccacagcctacatcctggtggtggcgggcactgtcgtcatggtgactggggtcttgggctgctgcgccaccttcaaggagcgtcggaacctgctgcgcctgtacttcatcctgctcctcatcatctttctgctggagatcatcgctggtatcctcgcctacgcctactaccagcagctgaacacggagctcaaggagaacctgaaggacaccatgaccaagcgctaccaccagccgggccatgaggctgtgaccagcgctgtggaccagctgcagcaggagttccactgctgtggcagcaacaactcacaggactggcgagacagtgagtggatccgctcacaggaggccggtggccgtgtggtcccagacagctgctgcaagacggtggtggctctttgtgggcagcgagaccatgcctccaacatctacaaggtggagggcggctgcatcaccaagttggagaccttcatccaggagcacctgagggtcattggggctgtggggatcggcattgcctgtgtgcaggtctttggcatgatcttcacgtgctgcctgtacaggagtctcaagctggagcactactgaccctgccttgggccttgctgctgctgcacccaactactgagctgagaccactgagtaccaggggctgggctccctgatgacacccaccctgtgccatcaccataacctctggggaccccaacctcagaggcagcttcaagtgccttttgctgcgcaccaatgcccagcaggggagggaggggggctggcggggcgaagtttggggggtgttttgtggggctccccagacacactctctgcctggtggtcagatgcaggttggaaggggccttgctgagtggcgcaaggccgagcgttcccagcagggggagaaacccttcacaccccaggcccttcaggaactggggccttgccttgcagccacatggccccatcccagttggggaagccaggtgagctctgacccttgggcctgggcctctgcccctcccaacccagccctcgtctccctcgacagcgcccctgctgtcttccccaccgcagtcacaccacccgaaatgccacgtggtcactgtgcactgccctgttcatgtgcctctgcggggcagggccttcctggttttgttcactgctgtacccagatgcctacaaccatccctgccacatacaggtgctcaataaacacttgtagagcagatggaaaaaaaaaaaaaaaaaaaaaaaaaa

SEQ ID NO: 7 - protein, 111 aa, Artificial sequence
  A polypeptide corresponding to amino acids 111-221 of human CD151
AYAYYQQLNTELKENLKDTMTKRYHQPGHEAVTSAVDQLQQEFHCCGSNNSQDWRDSEWIRSQEAGGRVVPDSCCKTVVALCGQRDHASNIYKVEGGCITKLETFIQEHLR

SEQ ID NO: 8 - DNA, 333 bp, Artificial sequence
  A polynucleotide coding for amino acids 111-221 of human CD151
gcctacgcctactaccagcagctgaacacggagctcaaggagaacctgaaggacaccatgaccaagcgctaccaccagccgggccatgaggctgtgaccagcgctgtggaccagctgcagcaggagttccactgctgtggcagcaacaactcacaggactggcgagacagtgagtggatccgctcacaggaggccggtggccgtgtggtcccagacagctgctgcaagacggtggtggctctttgtgggcagcgagaccatgcctccaacatctacaaggtggagggcggctgcatcaccaagttggagaccttcatccaggagcacctgagg

SEQ ID NO: 9 - protein, 19 aa, Artificial sequence
  A polypeptide corresponding to amino acids 40-58 of human CD151
WTLALKSDYISLLASGTYL

SEQ ID NO: 10 - DNA, 57 bp, Artificial sequence
  A polynucleotide coding for amino acids 40-58 of human CD151
tggacgctggccctcaagagtgactacatcagcctgctggcctcaggcacctacctg

SEQ ID NO: 11 - protein, 10 aa, Artificial sequence
  An examplary non-functional peptide analogue taken from amino acids 111-221 of human CD151
AYAYYQQLNT

SEQ ID NO: 12 - protein, 10 aa, Artificial sequence
  An examplary non-functional peptide analogue taken from amino acids 111-221 of human CD151
YAYYQQLNTE

SEQ ID NO: 13 - protein, 10 aa, Artificial sequence
  An examplary non-functional peptide analogue taken from amino acids 111-221 of human CD151
AYYQQLNTEL

SEQ ID NO: 14 - protein, 10 aa, Artificial sequence
  An examplary non-functional peptide analogue taken from amino acids 111-221 of human CD151
YYQQLNTELK

SEQ ID NO: 15 - protein, 10 aa, Artificial sequence
  An examplary non-functional peptide analogue taken from amino acids 111-221 of human CD151
YQQLNTELKE

SEQ ID NO: 16 - protein, 10 aa, Artificial sequence
  An examplary non-functional peptide analogue taken from amino acids 111-221 of human CD151
QQLNTELKEN

SEQ ID NO: 17 - protein, 10 aa, Artificial sequence
  An examplary non-functional peptide analogue taken from amino acids 111-221 of human CD151
QLNTELKENL

SEQ ID NO: 18 - protein, 10 aa, Artificial sequence
  An examplary non-functional peptide analogue taken from amino acids 111-221 of human CD151
LNTELKENLK

SEQ ID NO: 19 - protein, 10 aa, Artificial sequence
  An examplary non-functional peptide analogue taken from amino acids 111-221 of human CD151
NTELKENLKD

SEQ ID NO: 20 - protein, 10 aa, Artificial sequence
  An examplary non-functional peptide analogue taken from amino acids 111-221 of human CD151
TELKENLKDT

SEQ ID NO: 21 - protein, 10 aa, Artificial sequence
  An examplary non-functional peptide analogue taken from amino acids 111-221 of human CD151
ELKENLKDTM

SEQ ID NO: 22 - protein, 10 aa, Artificial sequence
  An examplary non-functional peptide analogue taken from amino acids 111-221 of human CD151
LKENLKDTMT

SEQ ID NO: 23 - protein, 10 aa, Artificial sequence
  An examplary non-functional peptide analogue taken from amino acids 111-221 of human CD151
KENLKDTMTK

SEQ ID NO: 24 - protein, 10 aa, Artificial sequence
  An examplary non-functional peptide analogue taken from amino acids 111-221 of human CD151
ENLKDTMTKR

SEQ ID NO: 25 - protein, 10 aa, Artificial sequence
  An examplary non-functional peptide analogue taken from amino acids 111-221 of human CD151
NLKDTMTKRY

SEQ ID NO: 26 - protein, 10 aa, Artificial sequence
  An examplary non-functional peptide analogue taken from amino acids 111-221 of human CD151
LKDTMTKRYH

SEQ ID NO: 27 - protein, 10 aa, Artificial sequence
  An examplary non-functional peptide analogue taken from amino acids 111-221 of human CD151
KDTMTKRYHQ

SEQ ID NO: 28 - protein, 10 aa, Artificial sequence
  An examplary non-functional peptide analogue taken from amino acids 111-221 of human CD151
DTMTKRYHQP

SEQ ID NO: 29 - protein, 10 aa, Artificial sequence
  An examplary non-functional peptide analogue taken from amino acids 111-221 of human CD151
TMTKRYHQPG

SEQ ID NO: 30 - protein, 10 aa, Artificial sequence
  An examplary non-functional peptide analogue taken from amino acids 111-221 of human CD151
MTKRYHQPGH

SEQ ID NO: 31 - protein, 10 aa, Artificial sequence
  An examplary non-functional peptide analogue taken from amino acids 111-221 of human CD151
TKRYHQPGHE

SEQ ID NO: 32 - protein, 10 aa, Artificial sequence
  An examplary non-functional peptide analogue taken from amino acids 111-221 of human CD151
KRYHQPGHEA

SEQ ID NO: 33 - protein, 10 aa, Artificial sequence
  An examplary non-functional peptide analogue taken from amino acids 111-221 of human CD151
RYHQPGHEAV

SEQ ID NO: 34 - protein, 10 aa, Artificial sequence
  An examplary non-functional peptide analogue taken from amino acids 111-221 of human CD151
YHQPGHEAVT

SEQ ID NO: 35 - protein, 10 aa, Artificial sequence
  An examplary non-functional peptide analogue taken from amino acids 111-221 of human CD151
HQPGHEAVTS

SEQ ID NO: 36 - protein, 10 aa, Artificial sequence
  An examplary non-functional peptide analogue taken from amino acids 111-221 of human CD151
QPGHEAVTSA

SEQ ID NO: 37 - protein, 10 aa, Artificial sequence
  An examplary non-functional peptide analogue taken from amino acids 111-221 of human CD151
PGHEAVTSAV

SEQ ID NO: 38 - protein, 10 aa, Artificial sequence
  An examplary non-functional peptide analogue taken from amino acids 111-221 of human CD151
GHEAVTSAVD

SEQ ID NO: 39 - protein, 10 aa, Artificial sequence
  An examplary non-functional peptide analogue taken from amino acids 111-221 of human CD151
HEAVTSAVDQ

SEQ ID NO: 40 - protein, 10 aa, Artificial sequence
  An examplary non-functional peptide analogue taken from amino acids 111-221 of human CD151
EAVTSAVDQL

SEQ ID NO: 41 - protein, 10 aa, Artificial sequence
  An examplary non-functional peptide analogue taken from amino acids 111-221 of human CD151
AVTSAVDQLQ

SEQ ID NO: 42 - protein, 10 aa, Artificial sequence
  An examplary non-functional peptide analogue taken from amino acids 111-221 of human CD151
VTSAVDQLQQ

SEQ ID NO: 43 - protein, 10 aa, Artificial sequence
  An examplary non-functional peptide analogue taken from amino acids 111-221 of human CD151
TSAVDQLQQE

SEQ ID NO: 44 - protein, 10 aa, Artificial sequence
  An examplary non-functional peptide analogue taken from amino acids 111-221 of human CD151
SAVDQLQQEF

SEQ ID NO: 45 - protein, 10 aa, Artificial sequence
  An examplary non-functional peptide analogue taken from amino acids 111-221 of human CD151
AVDQLQQEFH

SEQ ID NO: 46 - protein, 10 aa, Artificial sequence
  An examplary non-functional peptide analogue taken from amino acids 111-221 of human CD151
VDQLQQEFHC

SEQ ID NO: 47 - protein, 10 aa, Artificial sequence
  An examplary non-functional peptide analogue taken from amino acids 111-221 of human CD151
DQLQQEFHCC

SEQ ID NO: 48 - protein, 10 aa, Artificial sequence
  An examplary non-functional peptide analogue taken from amino acids 111-221 of human CD151
QLQQEFHCCG

SEQ ID NO: 49 - protein, 10 aa, Artificial sequence
  An examplary non-functional peptide analogue taken from amino acids 111-221 of human CD151
LQQEFHCCGS

SEQ ID NO: 50 - protein, 10 aa, Artificial sequence
  An examplary non-functional peptide analogue taken from amino acids 111-221 of human CD151
QQEFHCCGSN

SEQ ID NO: 51 - protein, 10 aa, Artificial sequence
  An examplary non-functional peptide analogue taken from amino acids 111-221 of human CD151
QEFHCCGSNN

SEQ ID NO: 52 - protein, 10 aa, Artificial sequence
  An examplary non-functional peptide analogue taken from amino acids 111-221 of human CD151
EFHCCGSNNS

SEQ ID NO: 53 - protein, 10 aa, Artificial sequence
  An examplary non-functional peptide analogue taken from amino acids 111-221 of human CD151
FHCCGSNNSQ

SEQ ID NO: 54 - protein, 10 aa, Artificial sequence
  An examplary non-functional peptide analogue taken from amino acids 111-221 of human CD151
HCCGSNNSQD

SEQ ID NO: 55 - protein, 10 aa, Artificial sequence
  An examplary non-functional peptide analogue taken from amino acids 111-221 of human CD151
CCGSNNSQDW

SEQ ID NO: 56 - protein, 10 aa, Artificial sequence
  An examplary non-functional peptide analogue taken from amino acids 111-221 of human CD151
CGSNNSQDWR

SEQ ID NO: 57 - protein, 10 aa, Artificial sequence
  An examplary non-functional peptide analogue taken from amino acids 111-221 of human CD151
GSNNSQDWRD

SEQ ID NO: 58 - protein, 10 aa, Artificial sequence
  An examplary non-functional peptide analogue taken from amino acids 111-221 of human CD151
SNNSQDWRDS

SEQ ID NO: 59 - protein, 10 aa, Artificial sequence
  An examplary non-functional peptide analogue taken from amino acids 111-221 of human CD151
NNSQDWRDSE

SEQ ID NO: 60 - protein, 10 aa, Artificial sequence
  An examplary non-functional peptide analogue taken from amino acids 111-221 of human CD151
NSQDWRDSEW

SEQ ID NO: 61 - protein, 10 aa, Artificial sequence
  An examplary non-functional peptide analogue taken from amino acids 111-221 of human CD151
SQDWRDSEWI

SEQ ID NO: 62 - protein, 10 aa, Artificial sequence
  An examplary non-functional peptide analogue taken from amino acids 111-221 of human CD151
QDWRDSEWIR

SEQ ID NO: 63 - protein, 10 aa, Artificial sequence
  An examplary non-functional peptide analogue taken from amino acids 111-221 of human CD151
DWRDSEWIRS

SEQ ID NO: 64 - protein, 10 aa, Artificial sequence
  An examplary non-functional peptide analogue taken from amino acids 111-221 of human CD151
WRDSEWIRSQ

SEQ ID NO: 65 - protein, 10 aa, Artificial sequence
  An examplary non-functional peptide analogue taken from amino acids 111-221 of human CD151
RDSEWIRSQE

SEQ ID NO: 66 - protein, 10 aa, Artificial sequence
  An examplary non-functional peptide analogue taken from amino acids 111-221 of human CD151
DSEWIRSQEA

SEQ ID NO: 67 - protein, 10 aa, Artificial sequence
  An examplary non-functional peptide analogue taken from amino acids 111-221 of human CD151
SEWIRSQEAG

SEQ ID NO: 68 - protein, 10 aa, Artificial sequence
  An examplary non-functional peptide analogue taken from amino acids 111-221 of human CD151
EWIRSQEAGG

SEQ ID NO: 69 - protein, 10 aa, Artificial sequence
  An examplary non-functional peptide analogue taken from amino acids 111-221 of human CD151
WIRSQEAGGR

SEQ ID NO: 70 - protein, 10 aa, Artificial sequence
  An examplary non-functional peptide analogue taken from amino acids 111-221 of human CD151
IRSQEAGGRV

SEQ ID NO: 71 - protein, 10 aa, Artificial sequence
  An examplary non-functional peptide analogue taken from amino acids 111-221 of human CD151
RSQEAGGRVV

SEQ ID NO: 72 - protein, 10 aa, Artificial sequence
  An examplary non-functional peptide analogue taken from amino acids 111-221 of human CD151
SQEAGGRVVP

SEQ ID NO: 73 - protein, 10 aa, Artificial sequence
  An examplary non-functional peptide analogue taken from amino acids 111-221 of human CD151
QEAGGRVVPD

SEQ ID NO: 74 - protein, 10 aa, Artificial sequence
  An examplary non-functional peptide analogue taken from amino acids 111-221 of human CD151
EAGGRVVPDS

SEQ ID NO: 75 - protein, 10 aa, Artificial sequence
  An examplary non-functional peptide analogue taken from amino acids 111-221 of human CD151
AGGRVVPDSC

SEQ ID NO: 76 - protein, 10 aa, Artificial sequence
  An examplary non-functional peptide analogue taken from amino acids 111-221 of human CD151
GGRVVPDSCC

SEQ ID NO: 77 - protein, 10 aa, Artificial sequence
  An examplary non-functional peptide analogue taken from amino acids 111-221 of human CD151
GRVVPDSCCK

SEQ ID NO: 78 - protein, 10 aa, Artificial sequence
  An examplary non-functional peptide analogue taken from amino acids 111-221 of human CD151
RVVPDSCCKT

SEQ ID NO: 79 - protein, 10 aa, Artificial sequence
  An examplary non-functional peptide analogue taken from amino acids 111-221 of human CD151
VVPDSCCKTV

SEQ ID NO: 80 - protein, 10 aa, Artificial sequence
  An examplary non-functional peptide analogue taken from amino acids 111-221 of human CD151
VPDSCCKTVV

SEQ ID NO: 81 - protein, 10 aa, Artificial sequence
  An examplary non-functional peptide analogue taken from amino acids 111-221 of human CD151
PDSCCKTVVA

SEQ ID NO: 82 - protein, 10 aa, Artificial sequence
  An examplary non-functional peptide analogue taken from amino acids 111-221 of human CD151
DSCCKTVVAL

SEQ ID NO: 83 - protein, 10 aa, Artificial sequence
  An examplary non-functional peptide analogue taken from amino acids 111-221 of human CD151
SCCKTVVALC

SEQ ID NO: 84 - protein, 10 aa, Artificial sequence
  An examplary non-functional peptide analogue taken from amino acids 111-221 of human CD151
CCKTVVALCG

SEQ ID NO: 85 - protein, 10 aa, Artificial sequence
  An examplary non-functional peptide analogue taken from amino acids 111-221 of human CD151
CKTVVALCGQ

SEQ ID NO: 86 - protein, 10 aa, Artificial sequence
  An examplary non-functional peptide analogue taken from amino acids 111-221 of human CD151
KTVVALCGQR

SEQ ID NO: 87 - protein, 10 aa, Artificial sequence
  An examplary non-functional peptide analogue taken from amino acids 111-221 of human CD151
TVVALCGQRD

SEQ ID NO: 88 - protein, 10 aa, Artificial sequence
  An examplary non-functional peptide analogue taken from amino acids 111-221 of human CD151
VVALCGQRDH

SEQ ID NO: 89 - protein, 10 aa, Artificial sequence
  An examplary non-functional peptide analogue taken from amino acids 111-221 of human CD151
VALCGQRDHA

SEQ ID NO: 90 - protein, 10 aa, Artificial sequence
  An examplary non-functional peptide analogue taken from amino acids 111-221 of human CD151
ALCGQRDHAS

SEQ ID NO: 91 - protein, 10 aa, Artificial sequence
  An examplary non-functional peptide analogue taken from amino acids 111-221 of human CD151
LCGQRDHASN

SEQ ID NO: 92 - protein, 10 aa, Artificial sequence
  An examplary non-functional peptide analogue taken from amino acids 111-221 of human CD151
CGQRDHASNI

SEQ ID NO: 93 - protein, 10 aa, Artificial sequence
  An examplary non-functional peptide analogue taken from amino acids 111-221 of human CD151
GQRDHASNIY

SEQ ID NO: 94 - protein, 10 aa, Artificial sequence
  An examplary non-functional peptide analogue taken from amino acids 111-221 of human CD151
QRDHASNIYK

SEQ ID NO: 95 - protein, 10 aa, Artificial sequence
  An examplary non-functional peptide analogue taken from amino acids 111-221 of human CD151
RDHASNIYKV

SEQ ID NO: 96 - protein, 10 aa, Artificial sequence
  An examplary non-functional peptide analogue taken from amino acids 111-221 of human CD151
DHASNIYKVE

SEQ ID NO: 97 - protein, 10 aa, Artificial sequence
  An examplary non-functional peptide analogue taken from amino acids 111-221 of human CD151
HASNIYKVEG

SEQ ID NO: 98 - protein, 10 aa, Artificial sequence
  An examplary non-functional peptide analogue taken from amino acids 111-221 of human CD151
ASNIYKVEGG

SEQ ID NO: 99 - protein, 10 aa, Artificial sequence
  An examplary non-functional peptide analogue taken from amino acids 111-221 of human CD151
SNIYKVEGGC

SEQ ID NO: 100 - protein, 10 aa, Artificial sequence
  An examplary non-functional peptide analogue taken from amino acids 111-221 of human CD151
NIYKVEGGCI

SEQ ID NO: 101 - protein, 10 aa, Artificial sequence
  An examplary non-functional peptide analogue taken from amino acids 111-221 of human CD151
IYKVEGGCIT

SEQ ID NO: 102 - protein, 10 aa, Artificial sequence
  An examplary non-functional peptide analogue taken from amino acids 111-221 of human CD151
YKVEGGCITK

SEQ ID NO: 103 - protein, 10 aa, Artificial sequence
  An examplary non-functional peptide analogue taken from amino acids 111-221 of human CD151
KVEGGCITKL

SEQ ID NO: 104 - protein, 10 aa, Artificial sequence
  An examplary non-functional peptide analogue taken from amino acids 111-221 of human CD151
VEGGCITKLE

SEQ ID NO: 105 - protein, 10 aa, Artificial sequence
  An examplary non-functional peptide analogue taken from amino acids 111-221 of human CD151
EGGCITKLET

SEQ ID NO: 106 - protein, 10 aa, Artificial sequence
  An examplary non-functional peptide analogue taken from amino acids 111-221 of human CD151
GGCITKLETF

SEQ ID NO: 107 - protein, 10 aa, Artificial sequence
  An examplary non-functional peptide analogue taken from amino acids 111-221 of human CD151
GCITKLETFI

SEQ ID NO: 108 - protein, 10 aa, Artificial sequence
  An examplary non-functional peptide analogue taken from amino acids 111-221 of human CD151
CITKLETFIQ

SEQ ID NO: 109 - protein, 10 aa, Artificial sequence
  An examplary non-functional peptide analogue taken from amino acids 111-221 of human CD151
ITKLETFIQE

SEQ ID NO: 110 - protein, 10 aa, Artificial sequence
  An examplary non-functional peptide analogue taken from amino acids 111-221 of human CD151
TKLETFIQEH

SEQ ID NO: 111 - protein, 10 aa, Artificial sequence
  An examplary non-functional peptide analogue taken from amino acids 111-221 of human CD151
KLETFIQEHL

SEQ ID NO: 112 - protein, 10 aa, Artificial sequence
  An examplary non-functional peptide analogue taken from amino acids 111-221 of human CD151
LETFIQEHLR

SEQ ID NO: 113 - protein, 10 aa, Artificial sequence
  An examplary non-functional peptide analogue taken from amino acids 40-58 of human CD151
WTLALKSDYI

SEQ ID NO: 114 - protein, 10 aa, Artificial sequence
  An examplary non-functional peptide analogue taken from amino acids 40-58 of human CD151
TLALKSDYIS

SEQ ID NO: 115 - protein, 10 aa, Artificial sequence
  An examplary non-functional peptide analogue taken from amino acids 40-58 of human CD151
LALKSDYISL

SEQ ID NO: 116 - protein, 10 aa, Artificial sequence
  An examplary non-functional peptide analogue taken from amino acids 40-58 of human CD151
ALKSDYISLL

SEQ ID NO: 117 - protein, 10 aa, Artificial sequence
  An examplary non-functional peptide analogue taken from amino acids 40-58 of human CD151
LKSDYISLLA

SEQ ID NO: 118 - protein, 10 aa, Artificial sequence
  An examplary non-functional peptide analogue taken from amino acids 40-58 of human CD151
KSDYISLLAS

SEQ ID NO: 119 - protein, 10 aa, Artificial sequence
  An examplary non-functional peptide analogue taken from amino acids 40-58 of human CD151
SDYISLLASG

SEQ ID NO: 120 - protein, 10 aa, Artificial sequence
  An examplary non-functional peptide analogue taken from amino acids 40-58 of human CD151
DYISLLASGT

SEQ ID NO: 121 - protein, 10 aa, Artificial sequence
  An examplary non-functional peptide analogue taken from amino acids 40-58 of human CD151
YISLLASGTY

SEQ ID NO: 122 - protein, 10 aa, Artificial sequence
  An examplary non-functional peptide analogue taken from amino acids 40-58 of human CD151
ISLLASGTYL

SEQ ID NO: 123 - DNA, 16 bp, Artificial sequence
  Single strand DNA oligonucleotide
cagtaacagtccgcct

SEQ ID NO: 124 - DNA, 16 bp, Artificial sequence
  Single strand DNA oligonucleotide
gtgacgttgacatccg

SEQ ID NO: 125 - DNA, 23 bp, Artificial sequence
  Single strand DNA oligonucleotide
taaagtggagggtggctgtatca

SEQ ID NO: 126 - DNA, 22 bp, Artificial sequence
  Single strand DNA oligonucleotide
aggtgaagatcatgccgaagac

SEQ ID NO: 127 - DNA, 762 bp, Homo sapiens
  misc_feature: Full-length human CD151
atgggtgagttcaacgagaagaagacaacatgtggcaccgtttgcctcaagtacctgctgtttacctacaattgctgcttctggctggctggcctggctgtcatggcagtgggcatctggacgctggccctcaagagtgactacatcagcctgctggcctcaggcacctacctggccacagcctacatcctggtggtggcgggcactgtcgtcatggtgactggggtcttgggctgctgcgccaccttcaaggagcgtcggaacctgctgcgcctgtacttcatcctgctcctcatcatctttctgctggagatcatcgctggtatcctcgcctacgcctactaccagcagctgaacacggagctcaaggagaacctgaaggacaccatgaccaagcgctaccaccagccgggccatgaggctgtgaccagcgctgtggaccagctgcagcaggagttccactgctgtggcagcaacaactcacaggactggcgagacagtgagtggatccgctcacaggaggccggtggccgtgtggtcccagacagctgctgcaagacggtggtggctctttgtgggcagcgagaccatgcctccaacatctacaaggtggagggcggctgcatcaccaagttggagaccttcatccaggagcacctgagggtcattggggctgtggggatcggcattgcctgtgtgcaggtctttggcatgatcttcacgtgctgcctgtacaggagtctcaagctggagcactactga

SEQ ID NO: 128 - DNA, 334 bp, Homo sapiens
  misc_feature: polynucleotide sequence coding for the extracellular domain of human CD151
gcctacgcctactaccagcagctgaacacggagctcaaggagaacctgaaggacaccatgaccaagcgctaccaccagccgggccatgaggctgtgaccagcgctgtggaccagctgcagcaggagttccactgctgtggcagcaacaactcacaggactggcgagacagtgagtggatccgctcacaggaggccggtggccgtgtggtcccagacagctgctgcaagacggtggtggctctttgtgggcagcgagaccatgcctccaacatctacaaggtggagggcggctgcatcaccaagttggagaccttcatccaggagcacctgaggg

SEQ ID NO: 129 - protein, 111 aa, Homo sapiens; identical to SEQ ID NO: 7
  misc_feature: Extracellular domain of human CD151 polypeptide sequence